(19) United States
(12) Patent Application Publication
(10) Pub. No.: US 2026/0008837 A1
(43) Pub. Date: Jan. 8, 2026

(54) BIOTECHNOLOGICAL PRODUCTION OF COLLAGEN PROTEINS AND BACTERIAL COLLAGEN-LIKE PROTEINS BY RECOMBINANT MICROORGANISMS

(71) Applicant: Evonik Operations GmbH, Essen (DE)

(72) Inventors: Steffen Schaffer, Herten (DE); Mirja Wessel, Bochum (DE); Linda Feldmann, Duelmen (DE)

(73) Assignee: Evonik Operations GmbH, Essen (DE)

(21) Appl. No.: 18/841,851

(22) PCT Filed: Feb. 28, 2023
(86) PCT No.: PCT/EP2023/054912

§ 371 Date: Aug. 27, 2024

(30) Foreign Application Priority Data
Mar. 1, 2022 (EP) 22159474.0

(51) Int. Cl. C07K 14/78; C07K 14/315; C12N 1/20; C12N 15/77; C12R 1/28

(52) U.S. Cl. CPC C07K 14/78; C07K 14/315; C12N 1/20; C12N 15/77; C07K 2319/02; C12R 2001/28

(57) ABSTRACT

Polynucleotides with an N-terminal signal sequence encode an amino acid sequence encoding a collagen protein or a bacterial collagen-like protein. Fusion products of collagen-like protein with various N-terminal signal peptides lead to increased production of collagen-like protein and secretion in a fermentative process for secreting bacterial collagen-like proteins in a host.

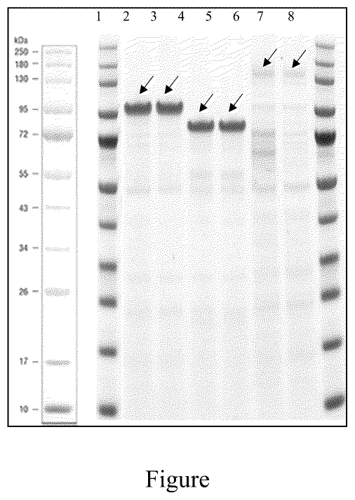

### CROSS-REFERENCE TO RELATED APPLICATIONS

This application is a National Stage entry under § 371 of International Application No. PCT/EP2023/054912, filed on Feb. 28, 2023, and which claims the benefit of priority to European Patent Application No. 22159474.0, filed on Mar. 1, 2022. The content of each of these applications is hereby incorporated by reference in its entirety.

### REFERENCE TO A SEQUENCE LISTING

The present application is accompanied by an XML file as a computer readable form containing the sequence listing entitled, “2025 Feb. 24 SeqList-006161USPCT-as-filed.xml”, created on Feb. 24, 2025, with a file size of 199,757 bytes, the content of which is hereby incorporated by reference in its entirety.

### BACKGROUND OF THE INVENTION

### Field of the Invention

The present invention relates to polynucleotides encoding an amino acid sequence encoding a collagen protein or a bacterial collagen-like protein, comprising an N-terminal signal sequence as well as a fermentative process for secreting collagen proteins and bacterial collagen-like proteins by a host into the fermentation broth.

Collagen-like proteins (CLPs) of bacterial origin (the most industrially relevant being the product of Streptomyces pyogenes) have considerably interesting mechanical properties, similar to those of higher eukaryotes' collagen proteins, without needing the complex maturing steps required for the eukaryotic counterparts. CLPs present a common structure: two alpha helixes, stabilizing each other, constitute a “V domain”, which is followed by a rod-like, structural collagen domain. After the collagen domain, typically a membrane anchor (GPI-like) is present at the C-terminal end of the protein.

### Description of Related Art

Expression of collagen-like proteins have been attempted in several systems, including Escherichia coli and Saccharomyces cerevisiae. For expression of scl2 in Escherichia coli (E. coli) (J. Biol. Chem. 277, 27312-27318) the construct of choice for such production carries a specific and necessary modification, in order to efficiently remove the potentially immunogenic V domain: such modification consists of a protease cleavage site typically inserted between the V domain and the collagen sequence. Due to this modification, the protein produced by the bacterial host must be extracted from the intracellular fraction and processed with a specific protease to remove the V domain. The mature protein, consisting of only the collagen-like domain, must be purified against the cleaved V domain, the whole intracellular protein content and the protease added to process the immature CLP. Such workflow greatly hinders the cost-effectiveness of the whole process, due to 1) the product of choice must be separated from the whole content of expression host cells, and 2) proteases are typically expensive enzymes.

### SUMMARY OF THE INVENTION

Therefore, it was an objective of the present invention to provide an improved process for the production of collagen proteins and CLPs, which is cost-effective and is applicable without the need to add specific proteases for cleavage of the domain.

Corynebacterium glutamicum (C. glutamicum) has been used in the past in many instances to produce heterologous proteins via secretion into the supernatant. It is exceptionally suitable for this task because of:
i) High capacity of the secretory apparatus to secrete proteins into the supernatant
ii) Almost complete lack of endogenous proteins secreted into the supernatant which would compete with heterologous proteins for the very same secretion apparatus
iii) Availability of a multitude of tools for its genetic manipulation
iv) A wealth of knowledge on its physiology and systems biology including genome sequence, gene expression, protein and metabolite abundance, etc.
v) More than 50 years of experience in industrial use and scale-up of this organism
vi) C. glutamicum being a safe host (GRAS notification) and thus no environmental or health concerns with respect to its industrial use

Nevertheless, efficient secretion of a given heterologous protein depends on many factors which are at least in part not well understood. This includes, but is not limited to:
I. Efficiency of gene expression i.e. transcription
II. Translation efficiency
III. Folding kinetics of the nascent polypeptide chain
IV. Three-dimensional structure of the folded polypeptide
V. Post-translational modifications of the polypeptide such as glycosylation or disulfide bonds
VI. Unfolding kinetics of the folded polypeptide by chaperones
VII. Presence of intracellular, membrane-bound or extracellular proteases, resulting in protein degradation
VIII. Choice of signal peptide (SP)
IX. Interaction of SP with signal recognition particle (SPR)
X. Interaction of the SPR-bound polypeptide chain with the Sec secretion apparatus
XI. Cleavage of the signal peptide cleavage site by signal peptidases

As a result, it is very difficult to rationally design a production strain for highly efficient secretory production of a given heterologous protein. Consequently, the individual factors are usually modulated by randomized approaches to create diversity with respect to the respective factor(s) followed by screening campaigns to sample this diversity.

This becomes obvious for instance by inspecting Table 1 in http://dx.doi.org/10.1016/j.jbiotec.2017.02.023. Protein concentrations in the fermentation broth as one parameter to describe the efficiency of secretion of heterologous proteins into the supernatant range from 0.5 mg to 5 g per L fermentation broth and thus span 4 orders of magnitude.

It was therefore not predictable and rather surprising that we were able to achieve high-level secretion of bacterial collagen-like proteins with Corynebacterium glutamicum using certain signal peptides. We therefore describe the use of these signal peptides for highly efficient production of collagen proteins and bacterial collagen-like proteins with Corynebacterium glutamicum and other bacterial strains.

It was even more surprising that choice of the signal peptide does not only impact the efficiency of the secretion of a given polypeptide but also the selectivity of the cleavage of the signal peptidase cleavage site by the cellular signal peptidases. This is highly important as the authenticity of the amino acid sequence of a given protein is highly important for many applications, most importantly in medical or pharmaceutical applications. The presence of variants of this protein with extra or missing amino acid residues at the N-terminus due to imprecise cleavage of the signal peptidase cleavage site by the cellular signal peptidases is highly problematic as these variants with extra or missing amino acid residues at the N-terminus need either to be removed by laborious purifications steps leading to higher manufacturing costs or it needs to be conformed that their presence in the formulation has no negative impact on performance of the protein or on the health of patients receiving treatments of which the protein formulation is part of. The latter is even more time-consuming and costly and sometimes impossible to confirm. The goal of the present invention therefore was, to provide an expression system for collagen proteins, including bacterial collagen-like proteins with enhanced secretion of the collagen proteins, and with a selective cleavage of the signal peptidase cleavage site by the cellular signal peptidases.

Therefore, the invention provides a novel fermentative process for secreting a collagen protein or a bacterial collagen-like protein, comprising an N-terminal signal sequence.

### DETAILED DESCRIPTION OF THE INVENTION

The invention relates to a polynucleotide encoding an amino acid sequence encoding a collagen protein or a bacterial collagen-like protein, comprising an N-terminal signal sequence that is at least ≥90% identical to one of the amino acid sequences selected from SEQ ID No: 6 to 66.

It was a surprising finding that specific fusion products of collagen-like protein with various N-terminal signal peptides lead to increased production of collagen-like protein and secretion into the fermentation medium.

It is preferred, when the N-terminal signal sequence is at least ≥92%, ≥94%, ≥96%, ≥97%, ≥98%, ≥99% or 100% identical to the amino acid sequence selected from SEQ ID No: 6 to 66.

In one embodiment, the polynucleotide according to the present invention may be a replicable nucleotide sequence encoding a collagen protein or a bacterial collagen-like protein from Streptococcus pyogenes, Glaesserella parasuis, Streptosporangium roseum, Chitinophaga varians, Hazenella sp., Paenibacillus larvae, Brevibacterium sp., Lacrimispora algidixylanolytica, Aquimarina sediminis, or from Brevibacillus reuszeri; preferably from Streptococcus pyogenes, Glaesserella parasuis or from Streptosporangium roseum.

In one embodiment, the polynucleotide according to the present invention is a replicable nucleotide sequence encoding the collagen-like protein from Streptococcus pyogenes.

More specifically, the polynucleotide may be a replicable nucleotide sequence encoding the collagen-like domain of the collagen-like protein from Streptococcus pyogenes. This refers to the collagen-like protein without the N-terminal V domain and without the C-terminal membrane anchor.

Therefore, it is preferred wherein the amino acid sequence encodes a bacterial collagen-like protein, comprising an N-terminal signal sequence, wherein the amino acid sequence is at least ≥90% identical to one of the amino acid sequences selected from SEQ ID No: 67 to 127.

The invention correspondingly also relates to a polynucleotide and nucleic acid molecules comprising such sequences and encoding polypeptide variants of SEQ ID No: 67 to 127, which contain one or more insertion(s) or deletion(s). Preferably, the polypeptide contains a maximum of 5, a maximum of 4, a maximum of 3, or a maximum of 2, insertions or deletions of amino acids.

In another preferred embodiment, the amino acid sequence encodes a bacterial collagen-like protein, comprising an N-terminal signal sequence, wherein the amino acid sequence is at least ≥90% identical to one of the amino acid sequences selected from SEQ ID No: SEQ ID No: 90, SEQ ID No: 101, SEQ ID No: 104 or SEQ ID No: 106.

The invention further relates to a polypeptide comprising an amino acid sequence encoded by the nucleotide sequence according to the invention.

The invention further relates to plasmids and vectors that comprise the nucleotide sequences according to the invention and optionally replicate in microorganisms of the genera Pichia, Corynebacterium, Pseudomonas or Escherichia or are suitable therefor. In a preferred configuration, the vector comprising the nucleotide sequences according to the present invention is suitable for replication in yeast of the genus Pichia pastoris.

The invention further relates to microorganisms of the genera Pichia, Corynebacterium, Pseudomonas or Escherichia that comprise the polynucleotides, vectors and polypeptides according to the invention.

Preferred microorganisms are Pichia pastoris, Brevibacillus choshinensis or Corynebacterium glutamicum.

The invention further relates to a microorganism according to the invention, characterized in that the polypeptide according to the invention is integrated in a chromosome. Homologous recombination permits, with use of the vectors according to the invention, the exchange of DNA sections on the chromosome for polynucleotides according to the invention which are transported into the cell by the vector. For efficient recombination between the ring-type DNA molecule of the vector and the target DNA on the chromosome, the DNA region that is to be exchanged containing the polynucleotide according to the invention is provided at the ends with nucleotide sequences homologous to the target site; these determine the site of integration of the vector and of exchange of the DNA.

The present invention provides a microorganism of the species P. pastoris, E. coli, P. putida or C. glutamicum comprising any of the nucleotide sequences or any of the polypeptides or any of the vectors from the description herein.

The microorganism may be a microorganism in which the nucleotide sequence is present in overexpressed form.

The microorganism may be characterized in that the microorganism has the capability of producing and secreting a fine chemical. The fine chemical being preferably a collagen protein or bacterial collagen-like protein.

Overexpression is taken to mean, generally, an increase in the intracellular concentration or activity of a ribonucleic acid, a protein (polypeptide) or an enzyme, compared with the starting strain (parent strain) or wild-type strain, if this is the starting strain. A starting strain (parent strain) is taken to mean the strain on which the measure leading to the overexpression was carried out.

In the overexpression, the methods of recombinant overexpression are preferred. These include all methods in which a microorganism is produced using a DNA molecule provided in vitro. Such DNA molecules comprise, for example, promoters, expression cassettes, genes, alleles, encoding regions etc. These are converted into the desired microorganism by methods of transformation, conjugation, transduction or like methods.

The extent of the expression or overexpression can be established by measuring the amount of the mRNA transcribed by the gene, by determining the amount of the polypeptide, and by determining the enzyme activity.

Disclosed is a fermentative process for producing a fine chemical comprising the following steps:
a) fermentation of a microorganism according to the present invention in a medium,
b) accumulation of the collagen protein or bacterial collagen-like protein in the medium, wherein a fermentation broth is obtained.

The use of such a process according to the invention leads, as shown in the Examples, to an extraordinary increase in product concentration and secretion of bacterial collagen-like protein compared with the respective starting strain.

The culture medium or fermentation medium that is to be used must appropriately satisfy the demands of the respective strains. Descriptions of culture media of various microorganisms are contained in the handbook “Manual of Methods for General Bacteriology” of the American Society for Bacteriology (Washington D.C., USA, 1981). The terms culture medium and fermentation medium or medium are mutually exchangeable.

In a preferred embodiment, the collagen protein or bacterial collagen-like protein is obtained in an amount of at least 100 mg/l, or at least 500 mg/l, or at least 1 g/l, or at least 5 g/l.

In another preferred embodiment, the purity of the collagen protein or bacterial collagen-like protein is at least 30%, or at least 50%, or at least 60%, or at least 70%, or at least 80%, or at least 90%, or at least 95%.

According to the present invention, the purity is defined as the amount of collagen-like protein with the correct amino acid sequence as defined above, in relation to the amount of total protein in the supernatant of the fermentation broth.

As carbon source, sugars and carbohydrates can be used, such as, e.g., glucose, sucrose, lactose, fructose, maltose, molasses, sucrose-containing solutions from beet sugar or sugar cane processing, starch, starch hydrolysate and cellulose, oils and fats, such as, for example, soybean oil, sunflower oil, groundnut oil and coconut fat, fatty acids, such as, for example, palmitic acid, stearic acid and linoleic acid, alcohols such as, for example, glycerol, methanol and ethanol, and organic acids, such as, for example, acetic acid or lactic acid.

As nitrogen source, organic nitrogen compounds such as peptones, yeast extract, meat extract, malt extract, corn-steep liquor, soybean meal and urea or inorganic compounds such as ammonium sulphate, ammonium chloride, ammonium phosphate, ammonium carbonate and ammonium nitrate can be used. The nitrogen sources can be used individually or as a mixture.

As phosphorus source, phosphoric acid, potassium dihydrogenphosphate or dipotassium hydrogenphosphate or the corresponding sodium-containing salts can be used.

The culture medium must, in addition, contain salts, for example in the form of chlorides or sulphates of metals such as, for example, sodium, potassium, magnesium, calcium and iron, such as, for example, magnesium sulphate or iron sulphate, which are necessary for growth. Finally, essential growth substances such as amino acids, for example homoserine and vitamins, for example thiamine, biotin or pantothenic acid, can be used in addition to the above-mentioned substances.

Said starting materials can be added to the culture in the form of a single batch or supplied in a suitable manner during the culturing.

Basic compounds such as sodium hydroxide, potassium hydroxide, ammonia or ammonia water, or acid compounds such as phosphoric acid or sulphuric acid, are used in a suitable manner for pH control of the culture. The pH is generally adjusted to 6.0 to 8.5, preferably 6.5 to 8. For control of foam development, antifoams can be used, such as, for example, polyglycol esters of fatty acids. For maintaining the stability of plasmids, suitable selectively acting substances such as, for example, antibiotics, can be added to the medium. The fermentation is preferably carried out under aerobic conditions. In order to maintain said aerobic conditions, oxygen or oxygen-containing gas mixtures such as, for example, air, are introduced into the culture. The use of liquids that are enriched with hydrogen peroxide is likewise possible. Optionally, the fermentation is carried out at superatmospheric pressure, for example at a superatmospheric pressure of 0.03 to 0.2 MPa. The temperature of the culture is usually 20° C. to 45° C., and preferably 25° C. to 40° C., particularly preferably 30° C. to 37° C. In the case of batch or fed-batch processes, the culturing is preferably continued until an amount sufficient for the measure of obtaining the desired organic chemical compound has formed. This goal is usually reached within 10 hours to 160 hours. In continuous processes, longer culture times are possible. Owing to the activity of the microorganisms, enrichment (accumulation) of the fine chemicals in the fermentation medium and/or in the cells of the microorganisms occurs.

Examples of suitable fermentation media may be found, inter alia, in patent documents U.S. Pat. Nos. 5,770,409, 5,990,350, 5,275,940, WO 2007/012078, U.S. Pat. No. 5,827,698, WO 2009/043803, U.S. Pat. No. 5,756,345 or U.S. Pat. No. 7,138,266; appropriate modifications may optionally be carried out to the requirements of the strains used.

The process may be characterized in that it is a process which is selected from the group consisting of batch process, fed-batch process, repetitive fed-batch process and continuous process.

The process may be further characterized in that the fine chemical or a liquid or solid fine chemical-containing product is obtained from the fine chemical-containing fermentation broth.

The performance of the processes or fermentation processes according to the invention with respect to one or more of the parameters selected from the group of concentration (compound formed per volume), yield (compound formed per carbon source consumed), volumetric productivity (compound formed per volume and time) and biomass-specific productivity (compound formed per cell dry mass or bio dry mass and time or compound formed per cell protein and time) or other process parameters and combinations thereof, is increased by at least 0.5%, at least 1%, at least 1.5% or at least 2%, based on processes or fermentation processes with microorganisms in which the promoter variant according to the invention is present.

Owing to the measures of the fermentation, a fermentation broth is obtained which contains the desired fine chemical, preferably amino acid or organic acid.

Then, a product in liquid or solid form that contains the fine chemical is provided or produced or obtained.

A fermentation broth is taken to mean, in a preferred embodiment, a fermentation medium or nutrient medium in which a microorganism was cultured for a certain time and at a certain temperature. The fermentation medium, or the media used during the fermentation, contains/contain all substances or components that ensure production of the desired compound and typically ensure growth and/or viability.

On completion of the fermentation, the resultant fermentation broth accordingly contains
a) the biomass (cell mass) of the microorganism resulting from growth of the cells of the microorganism,
b) the desired fine chemical formed in the course of the fermentation,
c) the organic by-products possibly formed in the course of the fermentation, and
d) the components of the fermentation medium used, or of the starting materials, that are not consumed by the fermentation, such as, for example, vitamins such as biotin, or salts such as magnesium sulphate.

The organic by-products include substances which are generated in addition to the respective desired compound by the microorganisms used in the fermentation and are possibly secreted.

The fermentation broth is withdrawn from the culture vessel or the fermentation container, optionally collected, and used for providing a product in liquid or solid form containing the fine chemical. The expression “obtaining the fine chemical-containing product” is also used therefor. In the simplest case, the fine chemical-containing fermentation broth withdrawn from the fermentation container is itself the product obtained.

By way of one or more of the measures selected from the group
a) partial (>0% to <80%) to complete (100%) or virtually complete (≥80%, ≥90%, ≥95%, ≥96%, ≥97%, ≥98%, ≥99%) removal of the water,
b) partial (>0% to <80%) to complete (100%) or virtually complete (≥80%, ≥90%, ≥95%, ≥96%, ≥97%, ≥98%, >99%) removal of the biomass, wherein this is optionally inactivated before the removal,
c) partial (>0% to <80%) to complete (100%) or virtually complete (≥80%, ≥90%, ≥95%, ≥96%, ≥97%, ≥98%, ≥99%, ≥99.3%, ≥99.7%) removal of the organic by-products formed in the course of the fermentation, and
d) partial (>0%) to complete (100%) or virtually complete (≥80%, ≥90%, ≥95%, ≥96%, ≥97%, ≥98%, ≥99%, ≥99.3%, >99.7%) removal of the components of the fermentation medium used or the starting materials that are not consumed by the fermentation,
a concentration or purification of the desired organic chemical compound is achieved from the fermentation broth. In this manner, products are isolated that have a desired content of the compound.

The partial (>0% to <80%) to complete (100%) or virtually complete (≥80% to <100%) removal of the water (measure a)) is also termed drying.

In a variant of the process, by complete or virtually complete removal of the water, the biomass, the organic by-products and the non-consumed components of the fermentation medium used, pure (≥80% by weight, ≥90% by weight) or high-purity (≥95% by weight, ≥97% by weight, ≥99% by weight) product forms of the desired organic chemical compound, preferably bacterial collagen-like protein, are successfully arrived at. For the measures according to a), b), c) or d), a great variety of technical instructions are available in the prior art.

In the case of processes for producing bacterial collagen-like protein processes are preferred in which products are obtained that do not contain any components of the fermentation broth. These products are used, in particular, in human medicine, in the pharmaceuticals industry, and in the food industry.

The process according to the invention serves for the fermentative production and secretion of collagen proteins and bacterial collagen-like proteins.

The invention finally relates to use of the microorganism according to the invention for the fermentative production and secretion of collagen proteins or bacterial collagen-like proteins.

### BRIEF DESCRIPTION OF THE DRAWINGS

The FIGURE: SDS-PAGE analysis of supernatants obtained from an expression culture. The collagen-like proteins were indicated by arrows. Lane 1: marker, lane 2 & 3: collagen-like domain 1 from Glaesserella parasuis (48.8 kDa), lane 4 & 5: collagen-like domain 2 from Glaesserella parasuis (48.2 kDa), lane 6 & 7: collagen-like domain from Streptosporangium roseum (70.6 kDa), lane 8: marker.

### EXAMPLES

The main steps for the production of a bacterial collagen-like protein with Corynebacterium glutamicum ATCC13032 can be summarized as follows:
I. Cloning the structural gene of the bacterial collagen-like protein in plasmid pXMJ19 under control of the IPTG-inducible tac promoter
II. Screening of a signal peptide library of ca. 170 signal peptides from C. glutamicum, B. subtilis and Brevibacillus choshinensis
III. Secretory production of bacterial collagen-like protein in the >170 strains harboring the expression plasmid with one of the signal peptides in microtiter plate format
IV. Quantitative analysis of fermentation broth from the MTP screening of the >170 strains using SDS-PAGE→semiquantitative protein concentration in the fermentation broth
V. Quantitative analysis of a subset (ca. 10 strains) of the fermentation broth from the MTP screening using HPLC→quantitative protein concentration in the fermentation broth and selectivity, i.e. proportion of the desired polypeptide relative to the total amount of polypeptides in the fermentation broth (MS-based identification of the amino acid sequence of the unwanted byproducts)

### Example 1: Construction of a C. glutamicum Expression Vector for the Streptococcus pyogenes Gene sclB_Spy Encoding for Bacterial Collagen-Like Protein

For the heterologous expression of the sclB_Spy gene (SEQ ID No:1) from Streptococcus pyogenes the plasmid pXMJ19 {Ptac}{SPnprE_Bs}[noV-sclB_Spy] was constructed. The sclB_Spy gene which encodes a bacterial collagen-like protein (SEQ ID No:2) was first fused with the signal peptide NprE from B. subtilis to enable the secretion of the collagen-like protein out of the cell (SEQ ID No:3). The sclB_Spy gene was cloned without the N-terminal “V domain” and without the C-terminal membrane anchor. The gene was cloned into the E. coli/C. glutamicum shuttle vector pXMJ19 (Jakoby et al., 1999). The expression of the sclB_Spy gene was under the control of the IPTG inducible promoter P_{tac} and downstream of the sclB_Spy gene a terminator sequence was located. The SPnprE_sclB_Spy fusion product (SEQ ID No:4) was ordered for gene synthesis from Eurofins Genomics Germany GmbH (Ebersberg, Germany) and cloned into the vector pXMJ19 using the restriction sites HindIII/EcoRI and NEBuilder® HiFi DNA Assembly Cloning Kit from New England BioLabs Inc., Ipswich, USA, Cat. No. E5520. The assembled product was transformed into 10-beta electrocompetent E. coli cells (New England BioLabs Inc., Ipswich, USA, Cat. No. C3020K). Procedure of cloning and transformation were carried out according to manufacturer's manual. The correct insertion of the target gene was checked by restriction analysis and the authenticity of the introduced DNA fragment was verified by DNA sequencing. The resulting expression vector was named pXMJ19 {Ptac}{SPnprE_Bs}[noV-sclB_Spy] (SEQ ID No:5, see table 1).

The C. glutamicum strain ATCC 13032 was transformed with the plasmid pXMJ19 {Ptac}{SPnprE_Bs}[noV-sclB_Spy] by means of electroporation and plated onto LB-agar plates supplemented with chloramphenicol (7.5 mg/l). Transformants were checked for the presence of the correct plasmid by plasmid preparation and analytic restriction analysis. The resulting strain was named C. glutamicum ATCC 13032 pXMJ19 {Ptac}{SPnprE_Bs}[noV-sclB_Spy] (see table 2).

### Example 2: Construction of C. glutamicum Expression Vectors for the Streptococcus pyogenes Gene sclB_Spy Encoding for Bacterial Collagen-Like Protein with Different Signal Peptides

For the replacement of the signal peptide sequence against alternatives 61 different plasmids were constructed. The different signal peptide sequences including the ribosome binding site in front of the signal peptide sequence were ordered for gene synthesis from Eurofins Genomics Germany GmbH (Ebersberg, Germany). For cloning the plasmid pXMJ19 {Ptac}{SPnprE_Bs}[noV-sclB_Spy] (SEQ ID No: 5) was cut with the restriction enzymes HindIII/XmaI. The original signal peptide sequence was removed and replaced by the alternative signal peptide sequences (SEQ ID No: 6-66) using NEBuilder® HiFi DNA Assembly Cloning Kit from New England BioLabs Inc., Ipswich, USA, Cat. No. E5520. For cloning each synthetic sequence contains upstream a 5′-overhang and ribosome binding site (5′-CAATTTCACACAGGAAACAGAATTAAGCTTGCATGCCTGCAGGAAGGAGATATAGAT-3′, SEQ ID No: 128) and downstream a 3′-overhang (5′-GGTAGTCCCGGGCTGCCAGGGCCCAGAGGGGAACAA-3′, SEQ ID No: 129). The synthetic constructs encode for fusion proteins (SEQ ID No: 67-127) consisting of a signal peptide from SEQ ID No: 6-66 and the bacterial collagen-like protein. The assembled product was transformed into 10-beta electrocompetent E. coli cells (New England BioLabs Inc., Ipswich, USA, Cat. No. C3020K). Procedure of cloning and transformation were carried out according to manufacturer's manual. The correct insertion of the target sequences was checked by restriction analysis and the authenticity of the introduced DNA fragments was verified by DNA sequencing. The resulting expression vectors were listed in table 1.

The C. glutamicum strain ATCC 13032 was transformed with plasmids 1 to 62 by means of electroporation and plated onto LB-agar plates supplemented with chloramphenicol (7.5 mg/l). Transformants were checked for the presence of the correct plasmid by plasmid preparation and analytic restriction analysis. The resulting strains were listed in table 2.

### Example 3: Production of Bacterial Collagen-Like Protein with C. glutamicum Derivatives

To produce the bacterial collagen-like protein a 96-deep well plate containing 1.8 ml BHI medium (GranuCult™ BHI (Brain Heart Infusion) broth, Merck, Darmstadt, Germany, Cat-No: 1.10493.0500) with chloramphenicol (7.5 mg/l) in each well was inoculated with 100 μl of a stock culture and incubated in a shaking incubator for 24 h at 33° C. and 1000 rpm. For the main culture, again a 96-deep well plate containing 1.8 ml BHI medium with chloramphenicol (7.5 mg/l) in each well was inoculated with the preculture to reach a start OD₆₀₀ of 0.1. The main culture was incubated for 48 h at 33° C. and 1000 rpm. After 5 h incubation the expression of the collagen-like gene was induced with 0.5 mM IPTG. At the end of cultivation, the cells were harvested, and supernatants were sterile-filtered with an 0.2 μm filter and stored at −20° C. before analysis. Collagen concentration of the strains was either analyzed via HPLC (see example 4) or via SDS-PAGE. The results provided in table 3 showed that 13 strains showed a collagen concentration of <100 mg/l, 38 strains showed a collagen concentration of 100-200 mg/l and 8 strains showed a collagen concentration of >201 mg/l. Exemplarily, three strains were listed which produced no collagen at all as representative for many other strains which showed no product secretion.

For selected strains the purity of the collagen-like protein was analyzed (see table 4). The strain C. glutamicum ATCC 13032 pXMJ19 {Ptac}{SPnprE_Bs}[noV-sclB_Spy] showed a purity of 34%. Beside the pure collagen-like protein the sample contained degradation products and also collagen variants where the signal peptide was not removed completely. Four strains showed a purity of >90% (SEQ ID No: 90, SEQ ID No: 101, SEQ ID No: 104 and SEQ ID No: 106), the other strains tested showed only a purity<40%.

### Example 4: HPLC-Based Quantification of Bacterial Collagen-Like Protein

Quantification of bacterial like-collagen protein was carried out by means of HPLC. If necessary, samples were diluted in Sodium-phosphate buffer (63 mM Na₂HPO₄, 19 mM NaH₂PO₄x2H₂O, PH 7.2). Before analysis samples have to be denatured. Therefore ˜1 ml diluted sample was introduced into a 1.5 ml reaction tube and incubated at 40° C. and 1000 rpm for 10 min. Subsequently, the samples were centrifuged for 2 min at 16100 g and 10° C. The supernatant was filled into a HPLC vial and the measurement had been started immediately.

For the detection and quantification of the bacterial collagen-like protein an UV detector (215 nm) was used. The measurement was carried out by means of Agilent Technologies 1200 Series (Santa Clara, Calif., USA) and a Bio SEC-5 column (300 Å, 4.6×300 mm, 5 μm, Agilent). The injection volume was 20 μl and the run time was 20 min at a flow rate of 0.4 ml/min. Mobile phase A: Sodium phosphate buffer, pH 7.2, 600 mM NaCl (63 mM Na₂HPO₄, 19 mM NaH₂PO₄x2H₂O, PH 7.2, 600 mM NaCl). The column temperature was 25° C. As reference material purified bacterial collagen-like protein was used whose identity and purity was checked by HPLC-MS/MS.

### Example 5: RP-HPLC Analysis for Determination of Collagen Purity

Determination of collagen purity was carried out by means of reversed phase HPLC. If necessary, samples were diluted in 0.1% (v/v) TFA in H₂O. Before analysis samples have to be denatured. Therefore ˜1 ml diluted sample was introduced into a 1.5 ml reaction tube and incubated at 40° C. and 1000 rpm for 10 min. Subsequently, the samples were centrifuged for 3 min at 16100 g and 10° C. The supernatant was filled into a HPLC vial and the measurement had been started immediately.

For the determination of the collagen purity an UV detector (215 nm) was used. The measurement was carried out by means of Agilent Technologies 1200 Series (Santa Clara, Calif., USA) and a Zorbax 300SB-C8 column (4.6×150 mm, 3.5 μm, Agilent). The injection volume was 20 μl and the run time was 40 min at a flow rate of 1 ml/min. Mobile phase A: aqueous 0.1% (v/V) TFA (trifluoracetic acid, solution); mobile phase B: 90% (v/V) acetonitrile, 10% 0.1% aqueous TFA (trifluoracetic acid, solution). The column temperature was 25° C. As reference material purified bacterial collagen-like protein was used whose identity and purity was checked by HPLC-MS/MS.

### Gradient:

| t [min] | [%] B | Flow [ml/min] | Max pressure [bar] |
|---|---|---|---|
| 0 | 10 | 1 | 450 |
| 8 | 10 | 1 | 450 |
| 20 | 32 | 1 | 450 |
| 25 | 100 | 1 | 450 |
| 30 | 100 | 1 | 450 |
| 35 | 10 | 1 | 450 |
| 40 | 10 | 1 | 450 |

### Example 6: Construction of a Vector for the Expression of the Bacterial Collagen-Like Domain 1 from Glaesserella parasuis

For the heterologous expression of the collagen-like domain 1 from Glaesserella parasuis (SEQ ID No: 130) the plasmid pXMJ19 {Ptac}{SP65}[clp1_Gp(co_Cg)] was constructed. The collagen-like domain was fused with the signal peptide SP65 from C. glutamicum ATCC 13032 (SEQ ID No:29) to enable the secretion of the collagen-like domain 1 out of the cell. The corresponding gene sequence clp1_Gp was codon-optimized for the expression in C. glutamicum and cloned into the E. coli/C. glutamicum shuttle vector pXMJ19 (Jakoby et al., 1999). The expression of the clp1_Gp gene was under the control of the IPTG inducible promoter P_{tac} and downstream of the gene a terminator sequence was located. The whole DNA sequence with overhangs for cloning (SEQ ID No: 131) was ordered for gene synthesis from Eurofins Genomics Germany GmbH (Ebersberg, Germany) and cloned into the vector pXMJ19 {Ptac}{SP65}[noV-sclB_Spy] (SEQ ID No:132) using the restriction sites XmaI/EcoRI and NEBuilder® HiFi DNA Assembly Cloning Kit from New England BioLabs Inc., Ipswich, USA, Cat. No. E5520. In this cloning step the sclB_Spy gene was replaced by clp1_Gp but the first six codons of sclB_Spy remain and were fused to the clp1_Gp gene. The assembled product encoding a fusion product consisting out of i) signal peptide SP65, ii) the first six amino acids of SclB_Spy and iii) Clp1_Gp (SEQ ID No:133) was transformed into 10-beta electrocompetent E. coli cells (New England BioLabs Inc., Ipswich, USA, Cat. No. C3020K). Procedure of cloning and transformation were carried out according to manufacturer's manual. The correct insertion of the target gene was checked by restriction analysis and the authenticity of the introduced DNA fragment was verified by DNA sequencing. The resulting expression vector was named pXMJ19 {Ptac}{SP65}[clp1_Gp(co_Cg)] (SEQ ID No: 134, see table 1).

The C. glutamicum strain ATCC 13032 was transformed with the plasmid pXMJ19 {Ptac}{SP65}[clp1_Gp(co_Cg)] by means of electroporation and plated onto LB-agar plates supplemented with chloramphenicol (7.5 mg/L). Transformants were checked for the presence of the correct plasmid by plasmid preparation and analytic restriction analysis. The resulting strain was named C. glutamicum ATCC 13032 pXMJ19 {Ptac}{SP65}[clp1_Gp(co_Cg)] (see table 2).

### Example 7: Construction of a Vector for the Expression of the Bacterial Collagen-Like Domain 2 from Glaesserella parasuis

For the heterologous expression of the collagen-like domain 2 from Glaesserella parasuis (SEQ ID No: 135) the plasmid pXMJ19 {Ptac}{SP65}[clp2_Gp(co_Cg)] was constructed. The collagen-like domain was fused with the signal peptide SP65 from C. glutamicum ATCC 13032 (SEQ ID No:29) to enable the secretion of the collagen-like domain 2 out of the cell. The corresponding gene sequence clp2_Gp was codon-optimized for the expression in C. glutamicum and cloned into the E. coli/C. glutamicum shuttle vector pXMJ19 (Jakoby et al., 1999). The expression of the clp2_Gp gene was under the control of the IPTG inducible promoter P_{tac} and downstream of the clp2_Gp gene a terminator sequence was located. The whole DNA sequence with overhangs for cloning (SEQ ID No: 136) was ordered for gene synthesis from Eurofins Genomics Germany GmbH (Ebersberg, Germany) and cloned into the vector pXMJ19 {Ptac}{SP65}[noV-sclB_Spy] (SEQ ID No:132) using the restriction sites XmaI/EcoRI and NEBuilder® HiFi DNA Assembly Cloning Kit from New England BioLabs Inc., Ipswich, USA, Cat. No. E5520. In this cloning step the sclB_Spy gene was replaced by clp2_Gp but the first six codons of sclB_Spy remain and were fused to the clp2_Gp gene. The assembled product encoding a fusion product consisting out of i) signal peptide SP65, ii) the first six amino acids of SclB_Spy and iii) Clp2_Gp (SEQ ID No:137) was transformed into 10-beta electrocompetent E. coli cells (New England BioLabs Inc., Ipswich, USA, Cat. No. C3020K). Procedure of cloning and transformation were carried out according to manufacturer's manual. The correct insertion of the target gene was checked by restriction analysis and the authenticity of the introduced DNA fragment was verified by DNA sequencing. The resulting expression vector was named pXMJ19 {Ptac}{SP65}[clp2_Gp(co_Cg)] (SEQ ID No:138, see table 1).

The C. glutamicum strain ATCC 13032 was transformed with the plasmid pXMJ19 {Ptac}{SP65}[clp2_Gp(co_Cg)] by means of electroporation and plated onto LB-agar plates supplemented with chloramphenicol (7.5 mg/L). Transformants were checked for the presence of the correct plasmid by plasmid preparation and analytic restriction analysis. The resulting strain was named C. glutamicum ATCC 13032 pXMJ19 {Ptac}{SP65}[clp2_Gp(co_Cg)] (see table 2).

### Example 8: Construction of a Vector for the Expression of the Bacterial Collagen-Like Domain from Streptosporangium roseum

For the heterologous expression of the collagen-like domain from Streptosporangium roseum (SEQ ID No: 139) the plasmid pXMJ19 {Ptac}{SP65}[clp_Sr(co_Cg)] was constructed. The collagen-like domain was fused with the signal peptide SP65 from C. glutamicum ATCC 13032 (SEQ ID No:29) to enable the secretion of the collagen-like domain out of the cell. The corresponding gene sequence clp_Sr was codon-optimized for the expression in C. glutamicum and cloned into the E. coli/C. glutamicum shuttle vector pXMJ19 (Jakoby et al., 1999). The expression of the clp_Sr gene was under the control of the IPTG inducible promoter P_{tac} and downstream of the clp_Sr gene a terminator sequence was located. The clp_Sr gene with overhangs for cloning (SEQ ID No: 140) was ordered for gene synthesis from Eurofins Genomics Germany GmbH (Ebersberg, Germany) and cloned into the vector pXMJ19 {Ptac}{SP65}[noV-sclB_Spy] (SEQ ID No:132) using the restriction sites XmaI/EcoRI and NEBuilder® HiFi DNA Assembly Cloning Kit from New England BioLabs Inc., Ipswich, USA, Cat. No. E5520. In this cloning step the sclB_Spy gene was replaced by clp_Sr but the first six codons of sclB_Spy remain and were fused to the clp_Sr gene. The assembled product encoding a fusion product consisting out of i) signal peptide SP65, ii) the first six amino acids of SclB_Spy and iii) Clp_Sr (SEQ ID No: 141) was transformed into 10-beta electrocompetent E. coli cells (New England BioLabs Inc., Ipswich, USA, Cat. No. C3020K). Procedure of cloning and transformation were carried out according to manufacturer's manual. The correct insertion of the target gene was checked by restriction analysis and the authenticity of the introduced DNA fragment was verified by DNA sequencing. The resulting expression vector was named pXMJ19 {Ptac}{SP65}[clp_Sr(co_Cg)] (SEQ ID No: 142, see table 1).

The C. glutamicum strain ATCC 13032 was transformed with the plasmid pXMJ19 {Ptac}{SP65}[clp_Sr(co_Cg)] by means of electroporation and plated onto LB-agar plates supplemented with chloramphenicol (7.5 mg/L). Transformants were checked for the presence of the correct plasmid by plasmid preparation and analytic restriction analysis. The resulting strain was named C. glutamicum ATCC 13032 pXMJ19 {Ptac}{SP65}[clp_Sr(co_Cg)] (see table 2).

### Example 9: Production of Bacterial Collagen-Like Protein with C. glutamicum Derivatives

To produce the bacterial collagen-like domains a 96-deep well plate containing 1.8 ml BHI medium (GranuCult™ BHI (Brain Heart Infusion) broth, Merck, Darmstadt, Germany, Cat-No: 1.10493.0500) with chloramphenicol (7.5 mg/L) in each well was inoculated with 100 μl of a stock culture and incubated in a shaking incubator for 24 h at 33° C. and 1000 rpm. For the main culture, again a 96-deep well plate containing 1.8 ml BHI medium with chloramphenicol (7.5 mg/L) in each well was inoculated with the preculture to reach a start OD₆₀₀ of 0.1. The main culture was incubated for 48 h at 33° C. and 1000 rpm. After 5 h incubation the expression of the collagen-like gene was induced with 0.5 mM IPTG. At the end of cultivation, the cells were harvested, and supernatants were sterile-filtered with an 0.2 μm filter and stored at −20° C. before analysis. Collagen production of the strains was analyzed via SDS-PAGE (see example 10).

### Example 10: SDS-Polyacrylamide Gel Electrophoresis for Detection of Bacterial Collagen-Like Proteins

Qualitative detection of bacterial like-collagen proteins was carried out by means of SDS polyacrylaminde gel electrophoresis (SDS-PAGE). 10 μl of the supernatants taken in example 9 were diluted 1:1 with NuPAGE™ LDS sample buffer (1×, ThermoFisher Scientific, Waltham, USA, Cat.-No. NP0007) and 2 μl DTT (0.5 M). Samples were incubated for 10 min at 90° C. and directly loaded on a NuPAGE 4-12% Bis-Tris Gel (ThermoFisher Scientific, Waltham, USA, Cat.-No NP0322BOX). The SDS-PAGE was carried out according to manufacturer's manual in NuPAGE™ MES SDS running buffer (1×, ThermoFisher Scientific, Waltham, USA, Cat.-No NP0002) at 200 V and for 40 min. After the electrophoresis the gels were incubated for 15 min in fixing solution (50% (v/v) ethanol, 7% (v/v) glacial acetic acid). In the next step the gels were incubated three times for 15 min in demineralized water and then stained for 1 h in GelCode® Blue Stain Reagent (ThermoFisher Scientific, Waltham, USA, Cat.-No. 24590). The gels were destained for 1 h by incubating in demineralized water. Before drying the gels were incubated for 15 min in drying solution (30% (v/v) ethanol, 15% (v/v) glycerol) and then dried with two sheets of cellophane. As shown in the FIGURE the three strains were able to produce a collagen-like protein.

TABLE 1
List of C. glutamicum expression plasmids

| Plasmid | SP No. | SP sequence |
|---|---|---|
| pXMJ19{Ptac}{SPnprE_Bs}[noV-sclB_Spy] | nprE | MGLGKKLSVAVAASFMSLSISLPGVQA |
| pXMJ19{Ptac}{SP29}[noV-sclB_Spy] | 29 | MRRSLRHGFTALLTTWALLLPTVAVA |
| pXMJ19{Ptac}{SP74[noV-sclB_Spy] | 74 | MSRISARTLAIALAGATAASLAVVPAATA |
| pXMJ19{Ptac}{SP75[noV-sclB_Spy] | 75 | MKHQYDVIVVGSGAGGLSAAVSA |
| pXMJ19{Ptac}{SP108[noV-sclB_Spy] | 108 | MRKKRVITCVMAASLTLGSLLPAGYASA |
| pXMJ19{Ptac}{SP8}[noV-sclB_Spy] | 8 | MASEKNLKLRTLAAAAGVLGVGAMSMLVAPQAAA |
| pXMJ19{Ptac}{SP9}[noV-sclB_Spy] | 9 | MKIKSVFLSTALSASLLLGITPPVLG |
| pXMJ19{Ptac}{SP12}[noV-sclB_Spy] | 12 | MQVFYNRAVTFTGMKTPRLLKILSAMVAVTGLLIPTVVPMAAADA |
| pXMJ19{Ptac}{SP13}[noV-sclB_Spy] | 13 | MVELSTRINPNERRFVVFIAAFLAMILVASGLATPAYA |
| pXMJ19{Ptac}{SP15}[noV-sclB_Spy] | 15 | MKSLPRFAPLITILALLVLVAIGGSALA |
| pXMJ19{Ptac}{SP16}[noV-sclB_Spy] | 16 | MSTTNSLTKLVASTVAAGVLGALALVPFASLSGVAVA |
| pXMJ19{Ptac}{SP17}[noV-sclB_Spy] | 17 | MKKFIAASTILALSLIGAPQAAAFS |
| pXMJ19{Ptac}{SP18}[noV-sclB_Spy] | 18 | MKRLSRAALAVVATTAVSFSALAVPAFA |
| pXMJ19{Ptac}{SP20}[noV-sclB_Spy] | 20 | MRNIVSTVAATIIASSLLLPTTAVTASA |
| pXMJ19{Ptac}{SP23}[noV-sclB_Spy] | 23 | MKLAPRMRMRSPKTFAALASLALVIGLGQVPIAQA |
| pXMJ19{Ptac}{SP32}[noV-sclB_Spy] | 32 | MKRMALIGSSLIISMGLITACGSAAAEPEAPAPTVTETVTATVTTTAKA |
| pXMJ19{Ptac}{SP35}[noV-sclB_Spy] | 35 | MKKKSFPIARVIGIGVLGIAGMGILLLWLAVTLSDPASPGA |
| pXMJ19{Ptac}{SP47}[noV-sclB_Spy] | 47 | MAQRKLASVIGAALAASAVLVGLMTPATA |
| pXMJ19{Ptac}{SP50}[noV-sclB_Spy] | 50 | MSLSNKAKGIVAVIIVAWIVAMAGMVAYA |
| pXMJ19{Ptac}{SP51}[noV-sclB_Spy] | 51 | MTRALIALAVSGALLSSMTPAVAQPQNPDDAAIAQA |
| pXMJ19{Ptac}{SP56}[noV-sclB_Spy] | 56 | MRKFRNTAIALVSAAAITLGGVTAATA |
| pXMJ19{Ptac}{SP58}[noV-sclB_Spy] | 58 | MSLRRSTLTLVTASAVALSVFTPVAQA |
| pXMJ19{Ptac}{SP60}[noV-sclB_Spy] | 60 | MRKGISRVLSVAVASSIGFGTVLTGTGIAAA |
| pXMJ19{Ptac}{SP64}[noV-sclB_Spy] | 64 | MNFSLVHLRENVRRVSVTVAIGAGALLISGPFFTAHTAEA |
| pXMJ19{Ptac}{SP65}[noV-sclB_Spy] | 65 | MQGMKRKMVAAALATALLFGAAPAHA |
| pXMJ19{Ptac}{SP68}[noV-sclB_Spy] | 68 | MKVVEVKKRNLLVAPLTASLVFCNLAVAANAVEVEA |
| pXMJ19{Ptac}{SP71}[noV-sclB_Spy] | 71 | MSFKRRSLAITCAITSSIVLAGISGTAAA |
| pXMJ19{Ptac}{SP79}[noV-sclB_Spy] | 79 | VKIKKSASALSRSMRIGIATITSTAMLGGVLVAVPAHPLLPTTAVAQA |
| pXMJ19{Ptac}{SP80}[noV-sclB_Spy] | 80 | MQSIRTTFRSLSRSAVIVGTVLALGIAGCSTASDEAT |
| pXMJ19{Ptac}{SP82}[noV-sclB_Spy] | 82 | MSVFTRAGEASRKLVALVVALATAAALMVVGQGTAQA |
| pXMJ19{Ptac}{SP83}[noV-sclB_Spy] | 83 | MKQVFLLFLAFLVLFPSNAHA |
| pXMJ19{Ptac}{SP84}[noV-sclB_Spy] | 84 | MKKIHMIPAALLIGSLALVSCAPSQPTQNAQTVSSVMT |
| pXMJ19{Ptac}{SP85}[noV-sclB_Spy] | 85 | MNISKLGLRIAVTATATTTALALGGTHMAVG |
| pXMJ19{Ptac}{SP87}[noV-sclB_Spy] | 87 | MKTIRTGMMTLAALAVLGTNVVSA |
| pXMJ19{Ptac}{SP88}[noV-sclB_Spy] | 88 | MKKVVNSVLASALALTVAPMAFA |
| pXMJ19{Ptac}{SP89}[noV-sclB_Spy] | 89 | MSISVRFKSLIALLMTVVFLLVPSSAFA |
| pXMJ19{Ptac}{SP90}[noV-sclB_Spy] | 90 | MKKRRVVNSVLLLLLLASALALTVAPMAFA |
| pXMJ19{Ptac}{SP91}[noV-sclB_Spy] | 91 | MFAKRFKTSLLPLFAGFLLLFHLVLAGPAAASA |
| pXMJ19{Ptac}{SP92}[noV-sclB_Spy] | 92 | MRKKTKNRLISSVLSTVVISSLLFPGAAGA |
| pXMJ19{Ptac}{SP93}[noV-sclB_Spy] | 93 | MKISMQKADFWKKAAISLLVFTMFFTLMMSETVFA |
| pXMJ19{Ptac}{SP94}[noV-sclB_Spy] | 94 | MKRLLSTLLIGIMLLTFAPSAFA |
| pXMJ19{Ptac}{SP95}[noV-sclB_Spy] | 95 | MKNMSCKLVVSVTLFFSFLTIGPLAHA |
| pXMJ19{Ptac}{SP96}[noV-sclB_Spy] | 96 | MKKVLMAFIICLSLILSVLAAPPSGAKA |
| pXMJ19{Ptac}{SP97}[noV-sclB_Spy] | 97 | MRSYIKVLTMCFLGLILFVPTALA |
| pXMJ19{Ptac}{SP102}[noV-sclB_Spy] | 102 | MKKGIIRFLLVSFVLFFALSTGITGVQAAPA |
| pXMJ19{Ptac}{SP103}[noV-sclB_Spy] | 103 | MKRRKFSSVVAAVLIFALIFSLFSPGTKAAA |
| pXMJ19{Ptac}{SP104}[noV-sclB_Spy] | 104 | MKTKTLFIFSAILTLSIFAPNETFA |
| pXMJ19{Ptac}{SP106}[noV-sclB_Spy] | 106 | MKRITINIITMFIAAAVISLTGTAEA |
| pXMJ19{Ptac}{SP109}[noV-sclB_Spy] | 109 | MKWMCSICCAAVLLAGGAAQA |
| pXMJ19{Ptac}{SP111}[noV-sclB_Spy] | 111 | MLTKRLLTIYIMLLGLIAWFPGAAQA |
| pXMJ19{Ptac}{SP112}[noV-sclB_Spy] | 112 | MRFTKVVGFLSVLGLAAVFPLTAQA |
| pXMJ19{Ptac}{SP115}[noV-sclB_Spy] | 115 | MKRFILVLSFLSIIVAYPIQTNA |
| pXMJ19{Ptac}{SP118}[noV-sclB_Spy] | 118 | MTKKAWFLPLVCVLLISGWLAPAASASA |
| pXMJ19{Ptac}{SP119}[noV-sclB_Spy] | 119 | MKRIRIPMTLALGAALTIAPLSFASA |
| pXMJ19{Ptac}{SP120}[noV-sclB_Spy] | 120 | MKKIVSILFMFGLVMGFSQFQPSTVFA |
| pXMJ19{Ptac}{SP128}[noV-sclB_Spy] | 128 | MVKSFRMKALIAGAAVAAAVSAGAVSDVPAAKVLQPTAAYA |
| pXMJ19{Ptac}{SP136}[noV-sclB_Spy] | 136 | MKRSISIFITCLLITLLTMGGMIASPASA |
| pXMJ19{Ptac}{SP140}[noV-sclB_Spy] | 140 | MKKLVLCVSILAVILSGVA |
| pXMJ19{Ptac}{SP144}[noV-sclB_Spy] | 144 | MKKKKTWKRFLHFSSAALAAGLIFTSAAPAEA |
| pXMJ19{Ptac}{SP145}[noV-sclB_Spy] | 145 | MKKSQYFIVFICFFVLFSVHPIAAAAA |
| pXMJ19{Ptac}{SP156}[noV-sclB_Spy] | 156 | MKKKIVAGLAVSAVVGSSMAAAPAEA |
| pXMJ19{Ptac}{SP159}[noV-sclB_Spy] | 159 | MFNRLFRVCFLAALIMAFTLPNSVYA |
| pXMJ19{Ptac}{SP65}[clp1_Gp(co_Cg)] | 65 | MQGMKRKMVAAALATALLFGAAPAHA |
| pXMJ19{Ptac}{SP65}[clp2_Gp(co_Cg)] | 56 | MQGMKRKMVAAALATALLFGAAPAHA |
| pXMJ19{Ptac}{SP65}[clp_Sr(co_Cg)] | 56 | MQGMKRKMVAAALATALLFGAAPAHA |

TABLE 2
List of plasmid-containing C. glutamicum strains

| | | Cloned | | |
|---|---|---|---|---|
| Plasmid | SP No. | gene | Recipient strain | Resulting strain |
| pXMJ19{Ptac}{SPnprE_Bs}[noV-sclB_Spy] | nprE | sclB_Spy | C. glutamicum ATCC 13032 | C. glutamicum ATCC 13032 |
| | | | | pXMJ19{Ptac}{SPnprE_Bs}[noV-sclB_Spy] |
| pXMJ19{Ptac}{SP29}[noV-sclB_Spy] | 29 | sclB_Spy | C. glutamicum ATCC 13032 | C. glutamicum ATCC 13032 |
| | | | | pXMJ19{Ptac}{SP29}[noV-sclB_Spy] |
| pXMJ19{Ptac}{SP74[noV-sclB_Spy] | 74 | sclB_Spy | C. glutamicum ATCC 13032 | C. glutamicum ATCC 13032 |
| | | | | pXMJ19{Ptac}{SP74}[noV-sclB_Spy] |
| pXMJ19{Ptac}{SP75[noV-sclB_Spy] | 75 | sclB_Spy | C. glutamicum ATCC 13032 | C. glutamicum ATCC 13032 |
| | | | | pXMJ19{Ptac}{SP75}[noV-sclB_Spy] |
| pXMJ19{Ptac}{SP108[noV-sclB_Spy] | 108 | sclB_Spy | C. glutamicum ATCC 13032 | C. glutamicum ATCC 13032 |
| | | | | pXMJ19{Ptac}{SP108}[noV-sclB_Spy] |
| pXMJ19{Ptac}{SP8}[noV-sclB_Spy] | 8 | sclB_Spy | C. glutamicum ATCC 13032 | C. glutamicum ATCC 13032 |
| | | | | pXMJ19{Ptac}{SP8}[noV-sclB_Spy] |
| pXMJ19{Ptac}{SP9}[noV-sclB_Spy] | 9 | sclB_Spy | C. glutamicum ATCC 13032 | C. glutamicum ATCC 13032 |
| | | | | pXMJ19{Ptac}{SP9}[noV-sclB_Spy] |
| pXMJ19{Ptac}{SP12}[noV-sclB_Spy] | 12 | sclB_Spy | C. glutamicum ATCC 13032 | C. glutamicum ATCC 13032 |
| | | | | pXMJ19{Ptac}{SP12}[noV-sclB_Spy] |
| pXMJ19{Ptac}{SP13}[noV-sclB_Spy] | 13 | sclB_Spy | C. glutamicum ATCC 13032 | C. glutamicum ATCC 13032 |
| | | | | pXMJ19{Ptac}{SP13}[noV-sclB_Spy] |
| pXMJ19{Ptac}{SP15}[noV-sclB_Spy] | 15 | sclB_Spy | C. glutamicum ATCC 13032 | C. glutamicum ATCC 13032 |
| | | | | pXMJ19{Ptac}{SP15}[noV-sclB_Spy] |
| pXMJ19{Ptac}{SP16}[noV-sclB_Spy] | 16 | sclB_Spy | C. glutamicum ATCC 13032 | C. glutamicum ATCC 13032 |
| | | | | pXMJ19{Ptac}{SP16}[noV-sclB_Spy] |
| pXMJ19{Ptac}{SP17}[noV-sclB_Spy] | 17 | sclB_Spy | C. glutamicum ATCC 13032 | C. glutamicum ATCC 13032 |
| | | | | pXMJ19{Ptac}{SP17}[noV-sclB_Spy] |
| pXMJ19{Ptac}{SP18}[noV-sclB_Spy] | 18 | sclB_Spy | C. glutamicum ATCC 13032 | C. glutamicum ATCC 13032 |
| | | | | pXMJ19{Ptac}{SP18}[noV-sclB_Spy] |
| pXMJ19{Ptac}{SP20}[noV-sclB_Spy] | 20 | sclB_Spy | C. glutamicum ATCC 13032 | C. glutamicum ATCC 13032 |
| | | | | pXMJ19{Ptac}{SP20}[noV-sclB_Spy] |
| pXMJ19{Ptac}{SP23}[noV-sclB_Spy] | 23 | sclB_Spy | C. glutamicum ATCC 13032 | C. glutamicum ATCC 13032 |
| | | | | pXMJ19{Ptac}{SP23}[noV-sclB_Spy] |
| pXMJ19{Ptac}{SP32}[noV-sclB_Spy] | 32 | sclB_Spy | C. glutamicum ATCC 13032 | C. glutamicum ATCC 13032 |
| | | | | pXMJ19{Ptac}{SP32}[noV-sclB_Spy] |
| pXMJ19{Ptac}{SP35}[noV-sclB_Spy] | 35 | sclB_Spy | C. glutamicum ATCC 13032 | C. glutamicum ATCC 13032 |
| | | | | pXMJ19{Ptac}{SP35}[noV-sclB_Spy] |
| pXMJ19{Ptac}{SP47}[noV-sclB_Spy] | 47 | sclB_Spy | C. glutamicum ATCC 13032 | C. glutamicum ATCC 13032 |
| | | | | pXMJ19{Ptac}{SP47}[noV-sclB_Spy] |
| pXMJ19{Ptac}{SP50}[noV-sclB_Spy] | 50 | sclB_Spy | C. glutamicum ATCC 13032 | C. glutamicum ATCC 13032 |
| | | | | pXMJ19{Ptac}{SP50}[noV-sclB_Spy] |
| pXMJ19{Ptac}{SP51}[noV-sclB_Spy] | 51 | sclB_Spy | C. glutamicum ATCC 13032 | C. glutamicum ATCC 13032 |
| | | | | pXMJ19{Ptac}{SP51}[noV-sclB_Spy] |
| pXMJ19{Ptac}{SP56}[noV-sclB_Spy] | 56 | sclB_Spy | C. glutamicum ATCC 13032 | C. glutamicum ATCC 13032 |
| | | | | pXMJ19{Ptac}{SP56}[noV-sclB_Spy] |
| pXMJ19{Ptac}{SP58}[noV-sclB_Spy] | 58 | sclB_Spy | C. glutamicum ATCC 13032 | C. glutamicum ATCC 13032 |
| | | | | pXMJ19{Ptac}{SP58}[noV-sclB_Spy] |
| pXMJ19{Ptac}{SP60}[noV-sclB_Spy] | 60 | sclB_Spy | C. glutamicum ATCC 13032 | C. glutamicum ATCC 13032 |
| | | | | pXMJ19{Ptac}{SP60}[noV-sclB_Spy] |
| pXMJ19{Ptac}{SP64}[noV-sclB_Spy] | 64 | sclB_Spy | C. glutamicum ATCC 13032 | C. glutamicum ATCC 13032 |
| | | | | pXMJ19{Ptac}{SP64}[noV-sclB_Spy] |
| pXMJ19{Ptac}{SP65}[noV-sclB_Spy] | 65 | sclB_Spy | C. glutamicum ATCC 13032 | C. glutamicum ATCC 13032 |
| | | | | pXMJ19{Ptac}{SP65}[noV-sclB_Spy] |
| pXMJ19{Ptac}{SP68}[noV-sclB_Spy] | 68 | sclB_Spy | C. glutamicum ATCC 13032 | C. glutamicum ATCC 13032 |
| | | | | pXMJ19{Ptac}{SP68}[noV-sclB_Spy] |
| pXMJ19{Ptac}{SP71}[noV-sclB_Spy] | 71 | sclB_Spy | C. glutamicum ATCC 13032 | C. glutamicum ATCC 13032 |
| | | | | pXMJ19{Ptac}{SP71}[noV-sclB_Spy] |
| pXMJ19{Ptac}{SP79}[noV-sclB_Spy] | 79 | sclB_Spy | C. glutamicum ATCC 13032 | C. glutamicum ATCC 13032 |
| | | | | pXMJ19{Ptac}{SP79}[noV-sclB_Spy] |
| pXMJ19{Ptac}{SP80}[noV-sclB_Spy] | 80 | sclB_Spy | C. glutamicum ATCC 13032 | C. glutamicum ATCC 13032 |
| | | | | pXMJ19{Ptac}{SP80}[noV-sclB_Spy] |
| pXMJ19{Ptac}{SP82}[noV-sclB_Spy] | 82 | sclB_Spy | C. glutamicum ATCC 13032 | C. glutamicum ATCC 13032 |
| | | | | pXMJ19{Ptac}{SP82}[noV-sclB_Spy] |
| pXMJ19{Ptac}{SP83}[noV-sclB_Spy] | 83 | sclB_Spy | C. glutamicum ATCC 13032 | C. glutamicum ATCC 13032 |
| | | | | pXMJ19{Ptac}{SP83}[noV-sclB_Spy] |
| pXMJ19{Ptac}{SP84}[noV-sclB_Spy] | 84 | sclB_Spy | C. glutamicum ATCC 13032 | C. glutamicum ATCC 13032 |
| | | | | pXMJ19{Ptac}{SP84}[noV-sclB_Spy] |
| pXMJ19{Ptac}{SP85}[noV-sclB_Spy] | 85 | sclB_Spy | C. glutamicum ATCC 13032 | C. glutamicum ATCC 13032 |
| | | | | pXMJ19{Ptac}{SP85}[noV-sclB_Spy] |
| pXMJ19{Ptac}{SP87}[noV-sclB_Spy] | 87 | sclB_Spy | C. glutamicum ATCC 13032 | C. glutamicum ATCC 13032 |
| | | | | pXMJ19{Ptac}{SP87}[noV-sclB_Spy] |
| pXMJ19{Ptac}{SP88}[noV-sclB_Spy] | 88 | sclB_Spy | C. glutamicum ATCC 13032 | C. glutamicum ATCC 13032 |
| | | | | pXMJ19{Ptac}{SP88}[noV-sclB_Spy] |
| pXMJ19{Ptac}{SP89}[noV-sclB_Spy] | 89 | sclB_Spy | C. glutamicum ATCC 13032 | C. glutamicum ATCC 13032 |
| | | | | pXMJ19{Ptac}{SP89}[noV-sclB_Spy] |
| pXMJ19{Ptac}{SP90}[noV-sclB_Spy] | 90 | sclB_Spy | C. glutamicum ATCC 13032 | C. glutamicum ATCC 13032 |
| | | | | pXMJ19{Ptac}{SP90}[noV-sclB_Spy] |
| pXMJ19{Ptac}{SP91}[noV-sclB_Spy] | 91 | sclB_Spy | C. glutamicum ATCC 13032 | C. glutamicum ATCC 13032 |
| | | | | pXMJ19{Ptac}{SP91}[noV-sclB_Spy] |
| pXMJ19{Ptac}{SP92}[noV-sclB_Spy] | 92 | sclB_Spy | C. glutamicum ATCC 13032 | C. glutamicum ATCC 13032 |
| | | | | pXMJ19{Ptac}{SP92}[noV-sclB_Spy] |
| pXMJ19{Ptac}{SP93}[noV-sclB_Spy] | 93 | sclB_Spy | C. glutamicum ATCC 13032 | C. glutamicum ATCC 13032 |
| | | | | pXMJ19{Ptac}{SP93}[noV-sclB_Spy] |
| pXMJ19{Ptac}{SP94}[noV-sclB_Spy] | 94 | sclB_Spy | C. glutamicum ATCC 13032 | C. glutamicum ATCC 13032 |
| | | | | pXMJ19{Ptac}{SP94}[noV-sclB_Spy] |
| pXMJ19{Ptac}{SP95}[noV-sclB_Spy] | 95 | sclB_Spy | C. glutamicum ATCC 13032 | C. glutamicum ATCC 13032 |
| | | | | pXMJ19{Ptac}{SP95}[noV-sclB_Spy] |
| pXMJ19{Ptac}{SP96}[noV-sclB_Spy] | 96 | sclB_Spy | C. glutamicum ATCC 13032 | C. glutamicum ATCC 13032 |
| | | | | pXMJ19{Ptac}{SP96}[noV-sclB_Spy] |
| pXMJ19{Ptac}{SP97}[noV-sclB_Spy] | 97 | sclB_Spy | C. glutamicum ATCC 13032 | C. glutamicum ATCC 13032 |
| | | | | pXMJ19{Ptac}{SP97}[noV-sclB_Spy] |
| pXMJ19{Ptac}{SP102}[noV-sclB_Spy] | 102 | sclB_Spy | C. glutamicum ATCC 13032 | C. glutamicum ATCC 13032 |
| | | | | pXMJ19{Ptac}{SP102}[noV-sclB_Spy] |
| pXMJ19{Ptac}{SP103}[noV-sclB_Spy] | 103 | sclB_Spy | C. glutamicum ATCC 13032 | C. glutamicum ATCC 13032 |
| | | | | pXMJ19{Ptac}{SP103}[noV-sclB_Spy] |
| pXMJ19{Ptac}{SP104}[noV-sclB_Spy] | 104 | sclB_Spy | C. glutamicum ATCC 13032 | C. glutamicum ATCC 13032 |
| | | | | pXMJ19{Ptac}{SP104}[noV-sclB_Spy] |
| pXMJ19{Ptac}{SP106}[noV-sclB_Spy] | 106 | sclB_Spy | C. glutamicum ATCC 13032 | C. glutamicum ATCC 13032 |
| | | | | pXMJ19{Ptac}{SP106}[noV-sclB_Spy] |
| pXMJ19{Ptac}{SP109}[noV-sclB_Spy] | 109 | sclB_Spy | C. glutamicum ATCC 13032 | C. glutamicum ATCC 13032 |
| | | | | pXMJ19{Ptac}{SP109}[noV-sclB_Spy] |
| pXMJ19{Ptac}{SP111}[noV-sclB_Spy] | 111 | sclB_Spy | C. glutamicum ATCC 13032 | C. glutamicum ATCC 13032 |
| | | | | pXMJ19{Ptac}{SP111}[noV-sclB_Spy] |
| pXMJ19{Ptac}{SP112}[noV-sclB_Spy] | 112 | sclB_Spy | C. glutamicum ATCC 13032 | C. glutamicum ATCC 13032 |
| | | | | pXMJ19{Ptac}{SP112}[noV-sclB_Spy] |
| pXMJ19{Ptac}{SP115}[noV-sclB_Spy] | 115 | sclB_Spy | C. glutamicum ATCC 13032 | C. glutamicum ATCC 13032 |
| | | | | pXMJ19{Ptac}{SP115}[noV-sclB_Spy] |
| pXMJ19{Ptac}{SP118}[noV-sclB_Spy] | 118 | sclB_Spy | C. glutamicum ATCC 13032 | C. glutamicum ATCC 13032 |
| | | | | pXMJ19{Ptac}{SP118}[noV-sclB_Spy] |
| pXMJ19{Ptac}{SP119}[noV-sclB_Spy] | 119 | slcB_Spy | C. glutamicum ATCC 13032 | C. glutamicum ATCC 13032 |
| | | | | pXMJ19{Ptac}{SP119}[noV-sclB_Spy] |
| pXMJ19{Ptac}{SP120}[noV-sclB_Spy] | 120 | slcB_Spy | C. glutamicum ATCC 13032 | C. glutamicum ATCC 13032 |
| | | | | pXMJ19{Ptac}{SP120}[noV-sclB_Spy] |
| pXMJ19{Ptac}{SP128}[noV-sclB_Spy] | 128 | slcB_Spy | C. glutamicum ATCC 13032 | C. glutamicum ATCC 13032 |
| | | | | pXMJ19{Ptac}{SP128}[noV-sclB_Spy] |
| pXMJ19{Ptac}{SP136}[noV-sclB_Spy] | 136 | slcB_Spy | C. glutamicum ATCC 13032 | C. glutamicum ATCC 13032 |
| | | | | pXMJ19{Ptac}{SP136}[noV-sclB_Spy] |
| pXMJ19{Ptac}{SP140}[noV-sclB_Spy] | 140 | slcB_Spy | C. glutamicum ATCC 13032 | C. glutamicum ATCC 13032 |
| | | | | pXMJ19{Ptac}{SP140}[noV-sclB_Spy] |
| pXMJ19{Ptac}{SP144}[noV-sclB_Spy] | 144 | slcB_Spy | C. glutamicum ATCC 13032 | C. glutamicum ATCC 13032 |
| | | | | pXMJ19{Ptac}{SP144}[noV-sclB_Spy] |
| pXMJ19{Ptac}{SP145}[noV-sclB_Spy] | 145 | slcB_Spy | C. glutamicum ATCC 13032 | C. glutamicum ATCC 13032 |
| | | | | pXMJ19{Ptac}{SP145}[noV-sclB_Spy] |
| pXMJ19{Ptac}{SP156}[noV-sclB_Spy] | 156 | slcB_Spy | C. glutamicum ATCC 13032 | C. glutamicum ATCC 13032 |
| | | | | pXMJ19{Ptac}{SP156}[noV-sclB_Spy] |
| pXMJ19{Ptac}{SP159}[noV-sclB_Spy] | 159 | slcB_Spy | C. glutamicum ATCC 13032 | C. glutamicum ATCC 13032 |
| | | | | pXMJ19{Ptac}{SP159}[noV-sclB_Spy] |
| pXMJ19{Ptac}{SP65}[clp1_Gp(co_Cg)] | 65 | clp1_Gp | C. glutamicum ATCC 13032 | C. glutamicum ATCC 13032 |
| | | | | pXMJ19{Ptac}{SP65}[clp1_Gp(co_Cg)] |
| pXMJ19{Ptac}{SP65}[clp2_Gp(co_Cg)] | 65 | clp2_Gp | C. glutamicum ATCC 13032 | C. glutamicum ATCC 13032 |
| | | | | pXMJ19{Ptac}{SP65}[clp2_Gp(co_Cg)] |
| pXMJ19{Ptac}{SP65}[clp_Sr(co_Cg)] | 65 | clp_Sr | C. glutamicum ATCC 13032 | C. glutamicum ATCC 13032 |
| | | | | pXMJ19{Ptac}{SP65}[clp_Sr(co_Cg)] |

TABLE 3
Collagen concentration analyzed via SEC analysis/SDS-PAGE

| Strain | Plasmid | SP No. | Collagen conc. [mg/l] |
|---|---|---|---|
| C. glutamicum ATCC 13032 | pXMJ19{Ptac}{SP29}[noV-sclB_Spy] | 29 | 0 |
| pXMJ19{Ptac}{SP29}[noV-sclB_Spy] | | | |
| C. glutamicum ATCC 13032 | pXMJ19{Ptac}{SP74[noV-sclB_Spy] | 74 | 0 |
| pXMJ19{Ptac}{SP74}[noV-sclB_Spy] | | | |
| C. glutamicum ATCC 13032 | pXMJ19{Ptac}{SP75[noV-sclB_Spy] | 75 | 0 |
| pXMJ19{Ptac}{SP75}[noV-sclB_Spy] | | | |
| C. glutamicum ATCC 13032 | pXMJ19{Ptac}{SP108[noV-sclB_Spy] | 108 | <100 |
| pXMJ19{Ptac}{SP108}[noV-sclB_Spy] | | | |
| C. glutamicum ATCC 13032 | pXMJ19{Ptac}{SP79}[noV-sclB_Spy] | 79 | <100 |
| pXMJ19{Ptac}{SP79}[noV-sclB_Spy] | | | |
| C. glutamicum ATCC 13032 | pXMJ19{Ptac}{SP80}[noV-sclB_Spy] | 80 | <100 |
| pXMJ19{Ptac}{SP80}[noV-sclB_Spy] | | | |
| C. glutamicum ATCC 13032 | pXMJ19{Ptac}{SP83}[noV-sclB_Spy] | 83 | <100 |
| pXMJ19{Ptac}{SP83}[noV-sclB_Spy] | | | |
| C. glutamicum ATCC 13032 | pXMJ19{Ptac}{SP90}[noV-sclB_Spy] | 90 | <100 |
| pXMJ19{Ptac}{SP90}[noV-sclB_Spy] | | | |
| C. glutamicum ATCC 13032 | pXMJ19{Ptac}{SP91}[noV-sclB_Spy] | 91 | <100 |
| pXMJ19{Ptac}{SP91}[noV-sclB_Spy] | | | |
| C. glutamicum ATCC 13032 | pXMJ19{Ptac}{SP97}[noV-sclB_Spy] | 97 | <100 |
| pXMJ19{Ptac}{SP97}[noV-sclB_Spy] | | | |
| C. glutamicum ATCC 13032 | pXMJ19{Ptac}{SP102}[noV-sclB_Spy] | 102 | <100 |
| pXMJ19{Ptac}{SP102}[noV-sclB_Spy] | | | |
| C. glutamicum ATCC 13032 | pXMJ19{Ptac}{SP103}[noV-sclB_Spy] | 103 | <100 |
| pXMJ19{Ptac}{SP103}[noV-sclB_Spy] | | | |
| C. glutamicum ATCC 13032 | pXMJ19{Ptac}{SP104}[noV-sclB_Spy] | 104 | <100 |
| pXMJ19{Ptac}{SP104}[noV-sclB_Spy] | | | |
| C. glutamicum ATCC 13032 | pXMJ19{Ptac}{SP111}[noV-sclB_Spy] | 111 | <100 |
| pXMJ19{Ptac}{SP111}[noV-sclB_Spy] | | | |
| C. glutamicum ATCC 13032 | pXMJ19{Ptac}{SP112}[noV-sclB_Spy] | 112 | <100 |
| pXMJ19{Ptac}{SP112}[noV-sclB_Spy] | | | |
| C. glutamicum ATCC 13032 | pXMJ19{Ptac}{SP156}[noV-sclB_Spy] | 156 | <100 |
| pXMJ19{Ptac}{SP156}[noV-sclB_Spy] | | | |
| C. glutamicum ATCC 13032 | pXMJ19{Ptac}{SPnprE_Bs}[noV-sclB_Spy] | nprE | 100-200 |
| pXMJ19{Ptac}{SPnprE_Bs}[noV-sclB_Spy] | | | |
| C. glutamicum ATCC 13032 | pXMJ19{Ptac}{SP8}[noV-sclB_Spy] | 8 | 100-200 |
| pXMJ19{Ptac}{SP8}[noV-sclB_Spy] | | | |
| C. glutamicum ATCC 13032 | pXMJ19{Ptac}{SP9}[noV-sclB_Spy] | 9 | 100-200 |
| pXMJ19{Ptac}{SP9}[noV-sclB_Spy] | | | |
| C. glutamicum ATCC 13032 | pXMJ19{Ptac}{SP12}[noV-sclB_Spy] | 12 | 100-200 |
| pXMJ19{Ptac}{SP12}[noV-sclB_Spy] | | | |
| C. glutamicum ATCC 13032 | pXMJ19{Ptac}{SP13}[noV-sclB_Spy] | 13 | 100-200 |
| pXMJ19{Ptac}{SP13}[noV-sclB_Spy] | | | |
| C. glutamicum ATCC 13032 | pXMJ19{Ptac}{SP15}[noV-sclB_Spy] | 15 | 100-200 |
| pXMJ19{Ptac}{SP15}[noV-sclB_Spy] | | | |
| C. glutamicum ATCC 13032 | pXMJ19{Ptac}{SP16}[noV-sclB_Spy] | 16 | 100-200 |
| pXMJ19{Ptac}{SP16}[noV-sclB_Spy] | | | |
| C. glutamicum ATCC 13032 | pXMJ19{Ptac}{SP17}[noV-sclB_Spy] | 17 | 100-200 |
| pXMJ19{Ptac}{SP17}[noV-sclB_Spy] | | | |
| C. glutamicum ATCC 13032 | pXMJ19{Ptac}{SP18}[noV-sclB_Spy] | 18 | 100-200 |
| pXMJ19{Ptac}{SP18}[noV-sclB_Spy] | | | |
| C. glutamicum ATCC 13032 | pXMJ19{Ptac}{SP20}[noV-sclB_Spy] | 20 | 100-200 |
| pXMJ19{Ptac}{SP20}[noV-sclB_Spy] | | | |
| C. glutamicum ATCC 13032 | pXMJ19{Ptac}{SP32}[noV-sclB_Spy] | 32 | 100-200 |
| pXMJ19{Ptac}{SP32}[noV-sclB_Spy] | | | |
| C. glutamicum ATCC 13032 | pXMJ19{Ptac}{SP35}[noV-sclB_Spy] | 35 | 100-200 |
| pXMJ19{Ptac}{SP35}[noV-sclB_Spy] | | | |
| C. glutamicum ATCC 13032 | pXMJ19{Ptac}{SP47}[noV-sclB_Spy] | 47 | 100-200 |
| pXMJ19{Ptac}{SP47}[noV-sclB_Spy] | | | |
| C. glutamicum ATCC 13032 | pXMJ19{Ptac}{SP50}[noV-sclB_Spy] | 50 | 100-200 |
| pXMJ19{Ptac}{SP50}[noV-sclB_Spy] | | | |
| C. glutamicum ATCC 13032 | pXMJ19{Ptac}{SP51}[noV-sclB_Spy] | 51 | 100-200 |
| pXMJ19{Ptac}{SP51}[noV-sclB_Spy] | | | |
| C. glutamicum ATCC 13032 | pXMJ19{Ptac}{SP56}[noV-sclB_Spy] | 56 | 100-200 |
| pXMJ19{Ptac}{SP56}[noV-sclB_Spy] | | | |
| C. glutamicum ATCC 13032 | pXMJ19{Ptac}{SP58}[noV-sclB_Spy] | 58 | 100-200 |
| pXMJ19{Ptac}{SP58}[noV-sclB_Spy] | | | |
| C. glutamicum ATCC 13032 | pXMJ19{Ptac}{SP60}[noV-sclB_Spy] | 60 | 100-200 |
| pXMJ19{Ptac}{SP60}[noV-sclB_Spy] | | | |
| C. glutamicum ATCC 13032 | pXMJ19{Ptac}{SP64}[noV-sclB_Spy] | 64 | 100-200 |
| pXMJ19{Ptac}{SP64}[noV-sclB_Spy] | | | |
| C. glutamicum ATCC 13032 | pXMJ19{Ptac}{SP68}[noV-sclB_Spy] | 68 | 100-200 |
| pXMJ19{Ptac}{SP68}[noV-sclB_Spy] | | | |
| C. glutamicum ATCC 13032 | pXMJ19{Ptac}{SP71}[noV-sclB_Spy] | 71 | 100-200 |
| pXMJ19{Ptac}{SP71}[noV-sclB_Spy] | | | |
| C. glutamicum ATCC 13032 | pXMJ19{Ptac}{SP82}[noV-sclB_Spy] | 82 | 100-200 |
| pXMJ19{Ptac}{SP82}[noV-sclB_Spy] | | | |
| C. glutamicum ATCC 13032 | pXMJ19{Ptac}{SP84}[noV-sclB_Spy] | 84 | 100-200 |
| pXMJ19{Ptac}{SP84}[noV-sclB_Spy] | | | |
| C. glutamicum ATCC 13032 | pXMJ19{Ptac}{SP85}[noV-sclB_Spy] | 85 | 100-200 |
| pXMJ19{Ptac}{SP85}[noV-sclB_Spy] | | | |
| C. glutamicum ATCC 13032 | pXMJ19{Ptac}{SP87}[noV-sclB_Spy] | 87 | 100-200 |
| pXMJ19{Ptac}{SP87}[noV-sclB_Spy] | | | |
| C. glutamicum ATCC 13032 | pXMJ19{Ptac}{SP88}[noV-sclB_Spy] | 88 | 100-200 |
| pXMJ19{Ptac}{SP88}[noV-sclB_Spy] | | | |
| C. glutamicum ATCC 13032 | pXMJ19{Ptac}{SP93}[noV-sclB_Spy] | 93 | 100-200 |
| pXMJ19{Ptac}{SP93}[noV-sclB_Spy] | | | |
| C. glutamicum ATCC 13032 | pXMJ19{Ptac}{SP95}[noV-sclB_Spy] | 95 | 100-200 |
| pXMJ19{Ptac}{SP95}[noV-sclB_Spy] | | | |
| C. glutamicum ATCC 13032 | pXMJ19{Ptac}{SP96}[noV-sclB_Spy] | 96 | 100-200 |
| pXMJ19{Ptac}{SP96}[noV-sclB_Spy] | | | |
| C. glutamicum ATCC 13032 | pXMJ19{Ptac}{SP109}[noV-sclB_Spy] | 109 | 100-200 |
| pXMJ19{Ptac}{SP109}[noV-sclB_Spy] | | | |
| C. glutamicum ATCC 13032 | pXMJ19{Ptac}{SP118}[noV-sclB_Spy] | 118 | 100-200 |
| pXMJ19{Ptac}{SP118}[noV-sclB_Spy] | | | |
| C. glutamicum ATCC 13032 | pXMJ19{Ptac}{SP119}[noV-sclB_Spy] | 119 | 100-200 |
| pXMJ19{Ptac}{SP119}[noV-sclB_Spy] | | | |
| C. glutamicum ATCC 13032 | pXMJ19{Ptac}{SP120}[noV-sclB_Spy] | 120 | 100-200 |
| pXMJ19{Ptac}{SP120}[noV-sclB_Spy] | | | |
| C. glutamicum ATCC 13032 | pXMJ19{Ptac}{SP128}[noV-sclB_Spy] | 128 | 100-200 |
| pXMJ19{Ptac}{SP128}[noV-sclB_Spy] | | | |
| C. glutamicum ATCC 13032 | pXMJ19{Ptac}{SP136}[noV-sclB_Spy] | 136 | 100-200 |
| pXMJ19{Ptac}{SP136}[noV-sclB_Spy] | | | |
| C. glutamicum ATCC 13032 | pXMJ19{Ptac}{SP140}[noV-sclB_Spy] | 140 | 100-200 |
| pXMJ19{Ptac}{SP140}[noV-sclB_Spy] | | | |
| C. glutamicum ATCC 13032 | pXMJ19{Ptac}{SP144}[noV-sclB_Spy] | 144 | 100-200 |
| pXMJ19{Ptac}{SP144}[noV-sclB_Spy] | | | |
| C. glutamicum ATCC 13032 | pXMJ19{Ptac}{SP145}[noV-sclB_Spy] | 145 | 100-200 |
| pXMJ19{Ptac}{SP145}[noV-sclB_Spy] | | | |
| C. glutamicum ATCC 13032 | pXMJ19{Ptac}{SP23}[noV-sclB_Spy] | 23 | 201-300 |
| pXMJ19{Ptac}{SP23}[noV-sclB_Spy] | | | |
| C. glutamicum ATCC 13032 | pXMJ19{Ptac}{SP89}[noV-sclB_Spy] | 89 | 201-300 |
| pXMJ19{Ptac}{SP89}[noV-sclB_Spy] | | | |
| C. glutamicum ATCC 13032 | pXMJ19{Ptac}{SP92}[noV-sclB_Spy] | 92 | 201-300 |
| pXMJ19{Ptac}{SP92}[noV-sclB_Spy] | | | |
| C. glutamicum ATCC 13032 | pXMJ19{Ptac}{SP94}[noV-sclB_Spy] | 94 | 201-300 |
| pXMJ19{Ptac}{SP94}[noV-sclB_Spy] | | | |
| C. glutamicum ATCC 13032 | pXMJ19{Ptac}{SP106}[noV-sclB_Spy] | 106 | 201-300 |
| pXMJ19{Ptac}{SP106}[noV-sclB_Spy] | | | |
| C. glutamicum ATCC 13032 | pXMJ19{Ptac}{SP115}[noV-sclB_Spy] | 115 | 201-300 |
| pXMJ19{Ptac}{SP115}[noV-sclB_Spy] | | | |
| C. glutamicum ATCC 13032 | pXMJ19{Ptac}{SP159}[noV-sclB_Spy] | 159 | 201-300 |
| pXMJ19{Ptac}{SP159}[noV-sclB_Spy] | | | |
| C. glutamicum ATCC 13032 | pXMJ19{Ptac}{SP65}[noV-sclB_Spy] | 65 | 301-400 |
| pXMJ19{Ptac}{SP65}[noV-sclB_Spy] | | | |

TABLE 4
Collagen purity analyzed via RP-HPLC

| Strain | Plasmid | CG SP No. | Purity [Area %] |
|---|---|---|---|
| C. glutamicum ATCC 13032 | pXMJ19{Ptac}{SPnprE_Bs}[noV-sclB_Spy] | nprE | 34 |
| pXMJ19{Ptac}{SPnprE_Bs}[noV-sclB_Spy] | | | |
| C. glutamicum ATCC 13032 | pXMJ19{Ptac}{SP65}[noV-sclB_Spy] | SP65 | 97 |
| pXMJ19{Ptac}{SP65}[noV-sclB_Spy] | | | |
| C. glutamicum ATCC 13032 | pXMJ19{Ptac}{SP106}[noV-sclB_Spy] | SP106 | 19 |
| pXMJ19{Ptac}{SP106}[noV-sclB_Spy] | | | |
| C. glutamicum ATCC 13032 | pXMJ19{Ptac}{SP115}[noV-sclB_Spy] | SP115 | 4 |
| pXMJ19{Ptac}{SP115}[noV-sclB_Spy] | | | |
| C. glutamicum ATCC 13032 | pXMJ19{Ptac}{SP89}[noV-sclB_Spy] | SP89 | 96 |
| pXMJ19{Ptac}{SP89}[noV-sclB_Spy] | | | |
| C. glutamicum ATCC 13032 | pXMJ19{Ptac}{SP92}[noV-sclB_Spy] | SP92 | 97 |
| pXMJ19{Ptac}{SP92}[noV-sclB_Spy] | | | |
| C. glutamicum ATCC 13032 | pXMJ19{Ptac}{SP94}[noV-sclB_Spy] | SP94 | 96 |
| pXMJ19{Ptac}{SP94}[noV-sclB_Spy] | | | |
| C. glutamicum ATCC 13032 | pXMJ19{Ptac}{SP51}[noV-sclB_Spy] | SP51 | 38 |
| pXMJ19{Ptac}{SP51}[noV-sclB_Spy] | | | |

### Sequences—DNA or Protein Sequence (AA)

SEQ ID No: 1 DNA sclB gene from S. pyogenes without V domain and membrane anchor
SEQ ID No: 2 AA bCL, collagen-like protein from S. pyogenes without V domain and membrane anchor
SEQ ID No: 3 AA fusion product consisting of signal peptide NprE from Bacillus subtilis and collagen-like domain from Streptococcus pyogenes
SEQ ID No: 4 DNA synthetic DNA fusion product consisting of signal peptide NprE and sclB_Spy gene with overhangs for cloning
SEQ ID No: 5 DNA plasmid pXMJ19 {Ptac}{SPnprE_Bs}[noV-sclB_Spy]
SEQ ID No: 6 AA Signal peptide No: 29
SEQ ID No: 7 AA Signal peptide No: 74
SEQ ID No: 8 AA Signal peptide No: 75
SEQ ID No: 9 AA Signal peptide No: 108
SEQ ID No: 10 AA Signal peptide No: 8
SEQ ID No: 11 AA Signal peptide No: 9
SEQ ID No: 12 AA Signal peptide No: 12
SEQ ID No: 13 AA Signal peptide No: 13
SEQ ID No: 14 AA Signal peptide No: 15
SEQ ID No: 15 AA Signal peptide No: 16
SEQ ID No: 16 AA Signal peptide No: 17
SEQ ID No: 17 AA Signal peptide No: 18
SEQ ID No: 18 AA Signal peptide No: 20
SEQ ID No: 19 AA Signal peptide No: 23
SEQ ID No: 20 AA Signal peptide No: 32
SEQ ID No 21 AA Signal peptide No: 35
SEQ ID No: 22 AA Signal peptide No: 47
SEQ ID No: 23 AA Signal peptide No: 50
SEQ ID No: 24 AA Signal peptide No: 51
SEQ ID No: 25 AA Signal peptide No: 56
SEQ ID No: 26 AA Signal peptide No: 58
SEQ ID No: 27 AA Signal peptide No: 60
SEQ ID No: 28 AA Signal peptide No: 64
SEQ ID No: 29 AA Signal peptide No: 65
SEQ ID No: 30 AA Signal peptide No: 68
SEQ ID No: 31 AA Signal peptide No: 71
SEQ ID No: 32 AA Signal peptide No: 79
SEQ ID No: 33 AA Signal peptide No: 80
SEQ ID No: 34 AA Signal peptide No: 82
SEQ ID No: 35 AA Signal peptide No: 83
SEQ ID No: 36 AA Signal peptide No: 84
SEQ ID No: 37 AA Signal peptide No: 85
SEQ ID No: 38 AA Signal peptide No: 87
SEQ ID No: 39 AA Signal peptide No: 88
SEQ ID No: 40 AA Signal peptide No: 89
SEQ ID No: 41 AA Signal peptide No: 90
SEQ ID No: 42 AA Signal peptide No: 91
SEQ ID No: 43 AA Signal peptide No: 92
SEQ ID No: 44 AA Signal peptide No: 93
SEQ ID No: 45 AA Signal peptide No: 94
SEQ ID No: 46 AA Signal peptide No: 95
SEQ ID No: 47 AA Signal peptide No: 96
SEQ ID No: 48 AA Signal peptide No: 97
SEQ ID No: 49 AA Signal peptide No: 102
SEQ ID No: 50 AA Signal peptide No: 103
SEQ ID No: 51 AA Signal peptide No: 104
SEQ ID No: 52 AA Signal peptide No: 106
SEQ ID No: 53 AA Signal peptide No: 109
SEQ ID No: 54 AA Signal peptide No: 111
SEQ ID No: 55 AA Signal peptide No: 112
SEQ ID No: 56 AA Signal peptide No: 115
SEQ ID No: 57 AA Signal peptide No: 118
SEQ ID No: 58 AA Signal peptide No: 119
SEQ ID No: 59 AA Signal peptide No: 120
SEQ ID No: 60 AA Signal peptide No: 128
SEQ ID No: 61 AA Signal peptide No: 136
SEQ ID No: 62 AA Signal peptide No: 140
SEQ ID No: 63 AA Signal peptide No: 144
SEQ ID No: 64 AA Signal peptide No: 145
SEQ ID No: 65 AA Signal peptide No: 156
SEQ ID No: 66 AA Signal peptide No: 159
SEQ ID No: 67 AA Fusion product of signal peptide 29 and collagen-like domain from Streptococcus pyogenes
SEQ ID No: 68 AA Fusion product of signal peptide No: 74 and collagen-like domain from Streptococcus pyogenes
SEQ ID No: 69 AA Fusion product of signal peptide No: 75 and collagen-like domain from Streptococcus pyogenes
SEQ ID No: 70 AA Fusion product of signal peptide No: 108 and collagen-like domain from Streptococcus pyogenes
SEQ ID No: 71 AA Fusion product of signal peptide No: 8 and collagen-like domain from Streptococcus pyogenes
SEQ ID No: 72 AA Fusion product of signal peptide No: 9 and collagen-like domain from Streptococcus pyogenes
SEQ ID No: 73 AA Fusion product of signal peptide No: 12 and collagen-like domain from Streptococcus pyogenes
SEQ ID No: 74 AA Fusion product of signal peptide No: 13 and collagen-like domain from Streptococcus pyogenes
SEQ ID No: 75 AA Fusion product of signal peptide No: 15 and collagen-like domain from Streptococcus pyogenes
SEQ ID No: 76 AA Fusion product of signal peptide No: 16 and collagen-like domain from Streptococcus pyogenes
SEQ ID No: 77 AA Fusion product of signal peptide No: 17 and collagen-like domain from Streptococcus pyogenes
SEQ ID No: 78 AA Fusion product of signal peptide No: 18 and collagen-like domain from Streptococcus pyogenes
SEQ ID No: 79 AA Fusion product of signal peptide No: 20 and collagen-like domain from Streptococcus pyogenes
SEQ ID No: 80 AA Fusion product of signal peptide No: 23 and collagen-like domain from Streptococcus pyogenes
SEQ ID No: 81 AA Fusion product of signal peptide No: 32 and collagen-like domain from Streptococcus pyogenes
SEQ ID No: 82 AA Fusion product of signal peptide No: 35 and collagen-like domain from Streptococcus pyogenes
SEQ ID No: 83 AA Fusion product of signal peptide No: 47 and collagen-like domain from Streptococcus pyogenes
SEQ ID No: 84 AA Fusion product of signal peptide No: 50 and collagen-like domain from Streptococcus pyogenes
SEQ ID No: 85 AA Fusion product of signal peptide No: 51 and collagen-like domain from Streptococcus pyogenes
SEQ ID No: 86 AA Fusion product of signal peptide No: 56 and collagen-like domain from Streptococcus pyogenes
SEQ ID No: 87 AA Fusion product of signal peptide No: 58 and collagen-like domain from Streptococcus pyogenes
SEQ ID No: 88 AA Fusion product of signal peptide No: 60 and collagen-like domain from Streptococcus pyogenes
SEQ ID No: 89 AA Fusion product of signal peptide No: 64 and collagen-like domain from Streptococcus pyogenes
SEQ ID No: 90 AA Fusion product of signal peptide No: 65 and collagen-like domain from Streptococcus pyogenes
SEQ ID No: 91 AA Fusion product of signal peptide No: 68 and collagen-like domain from Streptococcus pyogenes
SEQ ID No: 92 AA Fusion product of signal peptide No: 71 and collagen-like domain from Streptococcus pyogenes
SEQ ID No: 93 AA Fusion product of signal peptide No: 79 and collagen-like domain from Streptococcus pyogenes
SEQ ID No: 94 AA Fusion product of signal peptide No: 80 and collagen-like domain from Streptococcus pyogenes
SEQ ID No: 95 AA Fusion product of signal peptide No: 82 and collagen-like domain from Streptococcus pyogenes
SEQ ID No: 96 AA Fusion product of signal peptide No: 83 and collagen-like domain from Streptococcus pyogenes
SEQ ID No: 97 AA Fusion product of signal peptide No: 84 and collagen-like domain from Streptococcus pyogenes
SEQ ID No: 98 AA Fusion product of signal peptide No: 85 and collagen-like domain from Streptococcus pyogenes
SEQ ID No: 99 AA Fusion product of signal peptide No: 87 and collagen-like domain from Streptococcus pyogenes
SEQ ID No: 100 AA Fusion product of signal peptide No: 88 and collagen-like domain from Streptococcus pyogenes
SEQ ID No: 101 AA Fusion product of signal peptide No: 89 and collagen-like domain from Streptococcus pyogenes
SEQ ID No: 102 AA Fusion product of signal peptide No: 90 and collagen-like domain from Streptococcus pyogenes
SEQ ID No: 103 AA Fusion product of signal peptide No: 91 and collagen-like domain from Streptococcus pyogenes
SEQ ID No: 104 AA Fusion product of signal peptide No: 92 and collagen-like domain from Streptococcus pyogenes
SEQ ID No: 105 AA Fusion product of signal peptide No: 93 and collagen-like domain from Streptococcus pyogenes
SEQ ID No: 106 AA Fusion product of signal peptide No: 94 and collagen-like domain from Streptococcus pyogenes
SEQ ID No: 107 AA Fusion product of signal peptide No: 95 and collagen-like domain from Streptococcus pyogenes
SEQ ID No: 108 AA Fusion product of signal peptide No: 96 and collagen-like domain from Streptococcus pyogenes
SEQ ID No: 109 AA Fusion product of signal peptide No: 97 and collagen-like domain from Streptococcus pyogenes
SEQ ID No: 110 AA Fusion product of signal peptide No: 102 and collagen-like domain from Streptococcus pyogenes
SEQ ID No: 111 AA Fusion product of signal peptide No: 103 and collagen-like domain from Streptococcus pyogenes
SEQ ID No: 112 AA Fusion product of signal peptide No: 104 and collagen-like domain from Streptococcus pyogenes
SEQ ID No: 113 AA Fusion product of signal peptide No: 106 and collagen-like domain from Streptococcus pyogenes
SEQ ID No: 114 AA Fusion product of signal peptide No: 109 and collagen-like domain from Streptococcus pyogenes
SEQ ID No: 115 AA Fusion product of signal peptide No: 111 and collagen-like domain from Streptococcus pyogenes
SEQ ID No: 116 AA Fusion product of signal peptide No: 112 and collagen-like domain from Streptococcus pyogenes
SEQ ID No: 117 AA Fusion product of signal peptide No: 115 and collagen-like domain from Streptococcus pyogenes
SEQ ID No: 118 AA Fusion product of signal peptide No: 118 and collagen-like domain from Streptococcus pyogenes
SEQ ID No: 119 AA Fusion product of signal peptide No: 119 and collagen-like domain from Streptococcus pyogenes
SEQ ID No: 120 AA Fusion product of signal peptide No: 120 and collagen-like domain from Streptococcus pyogenes
SEQ ID No: 121 AA Fusion product of signal peptide No: 128 and collagen-like domain from Streptococcus pyogenes
SEQ ID No: 122 AA Fusion product of signal peptide No: 136 and collagen-like domain from Streptococcus pyogenes
SEQ ID No: 123 AA Fusion product of signal peptide No: 140 and collagen-like domain from Streptococcus pyogenes
SEQ ID No: 124 AA Fusion product of signal peptide No: 144 and collagen-like domain from Streptococcus pyogenes
SEQ ID No: 125 AA Fusion product of signal peptide No: 145 and collagen-like domain from Streptococcus pyogenes
SEQ ID No: 126 AA Fusion product of signal peptide No: 156 and collagen-like domain from Streptococcus pyogenes
SEQ ID No: 127 AA Fusion product of signal peptide No: 159 and collagen-like domain from Streptococcus pyogenes
SEQ ID No: 128 DNA synthetic sequence with upstream 5′-overhang and ribosome binding site
SEQ ID No: 129 DNA synthetic sequence with downstream 3′-overhang
SEQ ID No: 130 Clp1_Gp, collagen-like domain 1 from Glaesserella parasuis
SEQ ID No: 131 synthetic DNA fusion product consisting of the first codons of the sclB_Spy gene and the clp1_Gp gene including overhangs for cloning
SEQ ID No: 132 plasmid pXMJ19 {Ptac}{SP65}[noV-sclB_Spy]
SEQ ID No: 133 fusion product consisting of signal peptide SP65, the first six amino acids of SclB_Spy and Clp1_Gp
SEQ ID No: 134 plasmid pXMJ19 {Ptac}{SP65}[clp1_Gp(co_Cg)]
SEQ ID No: 135 Clp2_Gp, collagen-like domain 2 from Glaesserella parasuis
SEQ ID No: 136 synthetic DNA fusion product consisting of the first codons of the sclB_Spy gene and the clp2_Gp gene including overhangs for cloning
SEQ ID No: 137 fusion product consisting of signal peptide SP65, the first six amino acids of SclB_Spy and Clp2_Gp
SEQ ID No: 138 plasmid pXMJ19 {Ptac}{SP65}[clp2_Gp(co_Cg)]
SEQ ID No: 139 Clp_Sr, collagen-like domain from Streptosporangium roseum
SEQ ID No: 140 synthetic DNA fusion product consisting of the first codons of the sclB_Spy gene and the clp_Sr gene including overhangs for cloning
SEQ ID No: 141 fusion product consisting of signal peptide SP65, the first six amino acids of SclB_Spy and Clp_Sr
SEQ ID No: 142 plasmid pXMJ19 {Ptac}{SP65}[clp_Sr(co_Cg)]

1. A polynucleotide encoding an amino acid sequence encoding a collagen protein or a bacterial collagen-like protein, the polynucleotide comprising:
an N-terminal signal sequence that is at least ≥90% identical to one of an amino acid sequence selected from the group consisting of SEQ ID No: 6 to 66.

2. The polynucleotide according to claim 1, wherein the N-terminal signal sequence is at least >92% identical to the amino acid sequence selected from the group consisting of SEQ ID No: 6 to 66.

3. The polynucleotide according to claim 1, wherein the polynucleotide is a replicable nucleotide sequence encoding a collagen-like protein from Streptococcus pyogenes.

4. The polynucleotide according to claim 1, wherein the polynucleotide is a replicable nucleotide sequence encoding a collagen-like domain of a collagen-like protein from Streptococcus pyogenes.

5. The polynucleotide according to claim 1, wherein the amino acid sequence encodes a bacterial collagen-like protein, comprising:
an N-terminal signal sequence, wherein the amino acid sequence is at least ≥90% identical to one of an amino acid sequence selected from the group consisting of SEQ ID No: 67 to 127.

6. The polynucleotide according to claim 1, wherein the amino acid sequence encodes a bacterial collagen-like protein, comprising:
an N-terminal signal sequence, wherein the amino acid sequence is at least ≥90% identical to one of an amino acid sequence selected from the group consisting of SEQ ID No: 90, SEQ ID No: 101, SEQ ID No: 104, and SEQ ID No: 106.

7. A vector, comprising:
the polynucleotide according to claim 1.

8. A microorganism, comprising:
the polynucleotide according to claim 1.

9. The microorganism according to claim 8, wherein the microorganism is a microorganism of the genus Pichia, Brevibacillus, Bacillus, Escherichia or Corynebacterium.

10. The microorganism according to claim 9, wherein the polynucleotide is present in overexpressed form.

11. The microorganism according to claim 8, wherein the microorganism has a capability of secreting a collagen protein or bacterial collagen-like protein.

12. A fermentative process for secreting a collagen protein or bacterial collagen-like protein in a host, the process comprising:
a) fermentation of the microorganism according to claim 8 in a medium, and
b) accumulation of the collagen or bacterial collagen-like protein in the medium, wherein a fermentation broth is obtained.

13. The process according to claim 12, wherein the process is selected from the group consisting of batch process, fed-batch process, repetitive fed-batch process and continuous process.

14. The process according to claim 12, wherein the collagen protein or bacterial collagen-like protein is obtained in an amount of at least 100 mg/l.

15. The process according to claim 12, wherein a purity of the collagen protein or bacterial collagen-like protein is at least 30%.

16. A method, comprising:
fermentative production and/or secretion of collagen proteins and bacterial collagen-like proteins wherein the fermentative production and/or secretion of collagen proteins and bacterial collagen-like proteins is carried out with the microorganism according to claim 8.

17. The polynucleotide according to claim 2, wherein the N-terminal signal sequence is 100% identical to the amino acid sequence selected from the group consisting of SEQ ID No: 6 to 66.

18. The microorganism according to claim 9, wherein the microorganism is Pichia pastoris, Brevibacillus choshinensis or Corynebacterium glutamicum.

19. The process according to claim 14, wherein the collagen protein or bacterial collagen-like protein is obtained in an amount of at least 5 g/l.

20. The process according to claim 15, wherein the purity of the collagen protein or bacterial collagen-like protein is at least 95%.


SEQUENCE LISTING (142 sequences)

SEQ ID NO: 1 - DNA, 723 bp, Streptococcus pyogenes
  source 1..723: mol_type = other DNA
ggtagtcccgggctgccagggcccagaggggaacaaggaccaacaggtccaaccggacctgctggtccacgaggtctacaaggtctacaaggtctacaaggtgaaagaggggaacaaggaccaacaggtcccgctggtccacgaggtctacaaggtgaaagaggggaacaaggaccaacaggtctcgctggtaaagccggtgaagctggagccaaaggcgaaaccggccccgctggtccacagggtccacgtggtgaacaaggcccgcaaggtcttccaggtaaagatggtgaagctggtgctcaaggcccagcaggtccaatgggtcctgctggtgagcgaggtgaaaaaggagaacctggtacccaaggcgctaaaggtgatcgcggtgaaaccggtccagtaggtccacgtggtgagcgaggcgaagccggtcccgctggaaaagatggtgaacgtggtccagtaggtccagctggtaaggacggccaaaacggccaagatggtcttccaggtaaagacggtaaggacggccaaaacggtaaagatggtcttccaggtaaagacggtaaggacggccaaaacggtaaagatggtcttccaggtaaagacggtaaggacggtcaagatggtaaagacggcctcccaggtaaagacggtaaagatggcctcccaggtaaggacggtaaggacggtcaaccaggtaaaccgggtaaatattaa

SEQ ID NO: 2 - protein, 240 aa, Streptococcus pyogenes
GSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 3 - protein, 267 aa, Streptococcus pyogenes
MGLGKKLSVAVAASFMSLSISLPGVQAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 4 - DNA, 882 bp, synthetic construct
  source 1..882: mol_type = other DNA
tcacacaggaaacagaattaagcttgcatgcctgcaggaaggagatatagatatgggtttaggtaagaaattgtctgttgctgtcgctgcttcgtttatgagtttatcaatcagcctgccaggtgttcaggctggtagtcccgggctgccagggcccagaggggaacaaggaccaacaggtccaaccggacctgctggtccacgaggtctacaaggtctacaaggtctacaaggtgaaagaggggaacaaggaccaacaggtcccgctggtccacgaggtctacaaggtgaaagaggggaacaaggaccaacaggtctcgctggtaaagccggtgaagctggagccaaaggcgaaaccggccccgctggtccacagggtccacgtggtgaacaaggcccgcaaggtcttccaggtaaagatggtgaagctggtgctcaaggcccagcaggtccaatgggtcctgctggtgagcgaggtgaaaaaggagaacctggtacccaaggcgctaaaggtgatcgcggtgaaaccggtccagtaggtccacgtggtgagcgaggcgaagccggtcccgctggaaaagatggtgaacgtggtccagtaggtccagctggtaaggacggccaaaacggccaagatggtcttccaggtaaagacggtaaggacggccaaaacggtaaagatggtcttccaggtaaagacggtaaggacggccaaaacggtaaagatggtcttccaggtaaagacggtaaggacggtcaagatggtaaagacggcctcccaggtaaagacggtaaagatggcctcccaggtaaggacggtaaggacggtcaaccaggtaaaccgggtaaatattaagaattcagcttggctgttttggcgga

SEQ ID NO: 5 - DNA, 7384 bp, synthetic construct
  source 1..7384: mol_type = other DNA
agaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattaagcttgcatgcctgcaggaaggagatatagatatgggtttaggtaagaaattgtctgttgctgtcgctgcttcgtttatgagtttatcaatcagcctgccaggtgttcaggctggtagtcccgggctgccagggcccagaggggaacaaggaccaacaggtccaaccggacctgctggtccacgaggtctacaaggtctacaaggtctacaaggtgaaagaggggaacaaggaccaacaggtcccgctggtccacgaggtctacaaggtgaaagaggggaacaaggaccaacaggtctcgctggtaaagccggtgaagctggagccaaaggcgaaaccggccccgctggtccacagggtccacgtggtgaacaaggcccgcaaggtcttccaggtaaagatggtgaagctggtgctcaaggcccagcaggtccaatgggtcctgctggtgagcgaggtgaaaaaggagaacctggtacccaaggcgctaaaggtgatcgcggtgaaaccggtccagtaggtccacgtggtgagcgaggcgaagccggtcccgctggaaaagatggtgaacgtggtccagtaggtccagctggtaaggacggccaaaacggccaagatggtcttccaggtaaagacggtaaggacggccaaaacggtaaagatggtcttccaggtaaagacggtaaggacggccaaaacggtaaagatggtcttccaggtaaagacggtaaggacggtcaagatggtaaagacggcctcccaggtaaagacggtaaagatggcctcccaggtaaggacggtaaggacggtcaaccaggtaaaccgggtaaatattaagaattcagcttggctgttttggcggatgag

SEQ ID NO: 6 - protein, 26 aa, Corynebacterium glutamicum
  source 1..26: note = strain: Corynebacterium glutamicum ATCC 13032
MRRSLRHGFTALLTTWALLLPTVAVA

SEQ ID NO: 7 - protein, 29 aa, Corynebacterium glutamicum
  source 1..29: note = strain: Corynebacterium glutamicum ATCC 13032
MSRISARTLAIALAGATAASLAVVPAATA

SEQ ID NO: 8 - protein, 23 aa, Corynebacterium glutamicum
  source 1..23: note = strain: Corynebacterium glutamicum ATCC 13032
MKHQYDVIVVGSGAGGLSAAVSA

SEQ ID NO: 9 - protein, 28 aa, Bacillus subtilis
MRKKRVITCVMAASLTLGSLLPAGYASA

SEQ ID NO: 10 - protein, 34 aa, Corynebacterium glutamicum
  source 1..34: note = strain: Corynebacterium glutamicum ATCC 13032
MASEKNLKLRTLAAAAGVLGVGAMSMLVAPQAAA

SEQ ID NO: 11 - protein, 26 aa, Corynebacterium glutamicum
  source 1..26: note = strain: Corynebacterium glutamicum ATCC 13032
MKIKSVFLSTALSASLLLGITPPVLG

SEQ ID NO: 12 - protein, 45 aa, Corynebacterium glutamicum
  source 1..45: note = strain: Corynebacterium glutamicum ATCC 13032
MQVFYNRAVTFTGMKTPRLLKILSAMVAVTGLLIPTVVPMAAADA

SEQ ID NO: 13 - protein, 38 aa, Corynebacterium glutamicum
  source 1..38: note = strain: Corynebacterium glutamicum R
MVELSTRINPNERRFVVFIAAFLAMILVASGLATPAYA

SEQ ID NO: 14 - protein, 28 aa, Corynebacterium glutamicum
  source 1..28: note = strain: Corynebacterium glutamicum ATCC 13032
MKSLPRFAPLITILALLVLVAIGGSALA

SEQ ID NO: 15 - protein, 37 aa, Corynebacterium glutamicum
  source 1..37: note = strain: Corynebacterium glutamicum ATCC 13032
MSTTNSLTKLVASTVAAGVLGALALVPFASLSGVAVA

SEQ ID NO: 16 - protein, 25 aa, Corynebacterium glutamicum
  source 1..25: note = strain: Corynebacterium glutamicum R
MKKFIAASTILALSLIGAPQAAAFS

SEQ ID NO: 17 - protein, 28 aa, Corynebacterium glutamicum
  source 1..28: note = strain: Corynebacterium glutamicum ATCC 13032
MKRLSRAALAVVATTAVSFSALAVPAFA

SEQ ID NO: 18 - protein, 28 aa, Corynebacterium glutamicum
  source 1..28: note = strain: Corynebacterium glutamicum R
MRNIVSTVAATIIASSLLLPTTAVTASA

SEQ ID NO: 19 - protein, 35 aa, Corynebacterium glutamicum
  source 1..35: note = strain: Corynebacterium glutamicum ATCC 13032
MKLAPRMRMRSPKTFAALASLALVIGLGQVPIAQA

SEQ ID NO: 20 - protein, 49 aa, Corynebacterium glutamicum
  source 1..49: note = strain: Corynebacterium glutamicum ATCC 13032
MKRMALIGSSLIISMGLITACGSAAAEPEAPAPTVTETVTATVTTTAKA

SEQ ID NO: 21 - protein, 41 aa, Corynebacterium glutamicum
  source 1..41: note = strain: Corynebacterium glutamicum R
MKKKSFPIARVIGIGVLGIAGMGILLLWLAVTLSDPASPGA

SEQ ID NO: 22 - protein, 29 aa, Corynebacterium glutamicum
  source 1..29: note = strain: Corynebacterium glutamicum ATCC 13032
MAQRKLASVIGAALAASAVLVGLMTPATA

SEQ ID NO: 23 - protein, 29 aa, Corynebacterium glutamicum
  source 1..29: note = strain: Corynebacterium glutamicum ATCC 13032
MSLSNKAKGIVAVIIVAWIVAMAGMVAYA

SEQ ID NO: 24 - protein, 36 aa, Corynebacterium glutamicum
  source 1..36: note = strain: Corynebacterium glutamicum ATCC 13032
MTRALIALAVSGALLSSMTPAVAQPQNPDDAAIAQA

SEQ ID NO: 25 - protein, 27 aa, Corynebacterium glutamicum
  source 1..27: note = strain: Corynebacterium glutamicum ATCC 13032
MRKFRNTAIALVSAAAITLGGVTAATA

SEQ ID NO: 26 - protein, 27 aa, Corynebacterium glutamicum
  source 1..27: note = strain: Corynebacterium glutamicum ATCC 13032
MSLRRSTLTLVTASAVALSVFTPVAQA

SEQ ID NO: 27 - protein, 31 aa, Bacillus subtilis
MRKGISRVLSVAVASSIGFGTVLTGTGIAAA

SEQ ID NO: 28 - protein, 40 aa, Corynebacterium glutamicum
  source 1..40: note = strain: Corynebacterium glutamicum ATCC 13032
MNFSLVHLRENVRRVSVTVAIGAGALLISGPFFTAHTAEA

SEQ ID NO: 29 - protein, 26 aa, Corynebacterium glutamicum
  source 1..26: note = strain: Corynebacterium glutamicum ATCC 13032
MQGMKRKMVAAALATALLFGAAPAHA

SEQ ID NO: 30 - protein, 36 aa, Corynebacterium glutamicum
  source 1..36: note = strain: Corynebacterium glutamicum ATCC 13032
MKVVEVKKRNLLVAPLTASLVFCNLAVAANAVEVEA

SEQ ID NO: 31 - protein, 29 aa, Corynebacterium glutamicum
  source 1..29: note = strain: Corynebacterium glutamicum ATCC 13032
MSFKRRSLAITCAITSSIVLAGISGTAAA

SEQ ID NO: 32 - protein, 48 aa, Corynebacterium glutamicum
  source 1..48: note = strain: Corynebacterium glutamicum R
VKIKKSASALSRSMRIGIATITSTAMLGGVLVAVPAHPLLPTTAVAQA

SEQ ID NO: 33 - protein, 37 aa, Corynebacterium glutamicum
  source 1..37: note = strain: Corynebacterium glutamicum ATCC 13032
MQSIRTTFRSLSRSAVIVGTVLALGIAGCSTASDEAT

SEQ ID NO: 34 - protein, 37 aa, Corynebacterium glutamicum
  source 1..37: note = strain: Corynebacterium glutamicum ATCC 13032
MSVFTRAGEASRKLVALVVALATAAALMVVGQGTAQA

SEQ ID NO: 35 - protein, 21 aa, Corynebacterium glutamicum
  source 1..21: note = strain: Corynebacterium glutamicum R
MKQVFLLFLAFLVLFPSNAHA

SEQ ID NO: 36 - protein, 38 aa, Corynebacterium glutamicum
  source 1..38: note = strain: Corynebacterium glutamicum ATCC 13032
MKKIHMIPAALLIGSLALVSCAPSQPTQNAQTVSSVMT

SEQ ID NO: 37 - protein, 31 aa, Corynebacterium glutamicum
  source 1..31: note = strain: Corynebacterium glutamicum ATCC 13032
MNISKLGLRIAVTATATTTALALGGTHMAVG

SEQ ID NO: 38 - protein, 24 aa, Brevibacillus choshinensis
MKTIRTGMMTLAALAVLGTNVVSA

SEQ ID NO: 39 - protein, 23 aa, Brevibacillus choshinensis
MKKVVNSVLASALALTVAPMAFA

SEQ ID NO: 40 - protein, 28 aa, Brevibacillus choshinensis
MSISVRFKSLIALLMTVVFLLVPSSAFA

SEQ ID NO: 41 - protein, 30 aa, Brevibacillus choshinensis
MKKRRVVNSVLLLLLLASALALTVAPMAFA

SEQ ID NO: 42 - protein, 33 aa, Bacillus subtilis
MFAKRFKTSLLPLFAGFLLLFHLVLAGPAAASA

SEQ ID NO: 43 - protein, 30 aa, Bacillus subtilis
MRKKTKNRLISSVLSTVVISSLLFPGAAGA

SEQ ID NO: 44 - protein, 35 aa, Bacillus subtilis
MKISMQKADFWKKAAISLLVFTMFFTLMMSETVFA

SEQ ID NO: 45 - protein, 23 aa, Bacillus subtilis
MKRLLSTLLIGIMLLTFAPSAFA

SEQ ID NO: 46 - protein, 27 aa, Bacillus subtilis
MKNMSCKLVVSVTLFFSFLTIGPLAHA

SEQ ID NO: 47 - protein, 28 aa, Bacillus subtilis
MKKVLMAFIICLSLILSVLAAPPSGAKA

SEQ ID NO: 48 - protein, 24 aa, Bacillus subtilis
MRSYIKVLTMCFLGLILFVPTALA

SEQ ID NO: 49 - protein, 31 aa, Bacillus subtilis
MKKGIIRFLLVSFVLFFALSTGITGVQAAPA

SEQ ID NO: 50 - protein, 31 aa, Bacillus subtilis
MKRRKFSSVVAAVLIFALIFSLFSPGTKAAA

SEQ ID NO: 51 - protein, 25 aa, Bacillus subtilis
MKTKTLFIFSAILTLSIFAPNETFA

SEQ ID NO: 52 - protein, 26 aa, Bacillus subtilis
MKRITINIITMFIAAAVISLTGTAEA

SEQ ID NO: 53 - protein, 21 aa, Bacillus subtilis
MKWMCSICCAAVLLAGGAAQA

SEQ ID NO: 54 - protein, 26 aa, Bacillus subtilis
MLTKRLLTIYIMLLGLIAWFPGAAQA

SEQ ID NO: 55 - protein, 25 aa, Bacillus subtilis
MRFTKVVGFLSVLGLAAVFPLTAQA

SEQ ID NO: 56 - protein, 23 aa, Bacillus subtilis
MKRFILVLSFLSIIVAYPIQTNA

SEQ ID NO: 57 - protein, 28 aa, Bacillus subtilis
MTKKAWFLPLVCVLLISGWLAPAASASA

SEQ ID NO: 58 - protein, 26 aa, Bacillus subtilis
MKRIRIPMTLALGAALTIAPLSFASA

SEQ ID NO: 59 - protein, 27 aa, Bacillus subtilis
MKKIVSILFMFGLVMGFSQFQPSTVFA

SEQ ID NO: 60 - protein, 41 aa, Bacillus subtilis
MVKSFRMKALIAGAAVAAAVSAGAVSDVPAAKVLQPTAAYA

SEQ ID NO: 61 - protein, 29 aa, Bacillus subtilis
MKRSISIFITCLLITLLTMGGMIASPASA

SEQ ID NO: 62 - protein, 19 aa, Bacillus subtilis
MKKLVLCVSILAVILSGVA

SEQ ID NO: 63 - protein, 32 aa, Bacillus subtilis
MKKKKTWKRFLHFSSAALAAGLIFTSAAPAEA

SEQ ID NO: 64 - protein, 27 aa, Bacillus subtilis
MKKSQYFIVFICFFVLFSVHPIAAAAA

SEQ ID NO: 65 - protein, 26 aa, Bacillus subtilis
MKKKIVAGLAVSAVVGSSMAAAPAEA

SEQ ID NO: 66 - protein, 26 aa, Bacillus subtilis
MFNRLFRVCFLAALIMAFTLPNSVYA

SEQ ID NO: 67 - protein, 266 aa, synthetic construct
MRRSLRHGFTALLTTWALLLPTVAVAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 68 - protein, 269 aa, synthetic construct
MSRISARTLAIALAGATAASLAVVPAATAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 69 - protein, 263 aa, synthetic construct
MKHQYDVIVVGSGAGGLSAAVSAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 70 - protein, 268 aa, synthetic construct
MRKKRVITCVMAASLTLGSLLPAGYASAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 71 - protein, 274 aa, synthetic construct
MASEKNLKLRTLAAAAGVLGVGAMSMLVAPQAAAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 72 - protein, 266 aa, synthetic construct
MKIKSVFLSTALSASLLLGITPPVLGGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 73 - protein, 285 aa, synthetic construct
MQVFYNRAVTFTGMKTPRLLKILSAMVAVTGLLIPTVVPMAAADAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 74 - protein, 278 aa, synthetic construct
MVELSTRINPNERRFVVFIAAFLAMILVASGLATPAYAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 75 - protein, 268 aa, synthetic construct
MKSLPRFAPLITILALLVLVAIGGSALAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 76 - protein, 277 aa, synthetic construct
MSTTNSLTKLVASTVAAGVLGALALVPFASLSGVAVAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 77 - protein, 265 aa, synthetic construct
MKKFIAASTILALSLIGAPQAAAFSGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 78 - protein, 268 aa, synthetic construct
MKRLSRAALAVVATTAVSFSALAVPAFAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 79 - protein, 268 aa, synthetic construct
MRNIVSTVAATIIASSLLLPTTAVTASAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 80 - protein, 275 aa, synthetic construct
MKLAPRMRMRSPKTFAALASLALVIGLGQVPIAQAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 81 - protein, 289 aa, synthetic construct
MKRMALIGSSLIISMGLITACGSAAAEPEAPAPTVTETVTATVTTTAKAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 82 - protein, 281 aa, synthetic construct
MKKKSFPIARVIGIGVLGIAGMGILLLWLAVTLSDPASPGAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 83 - protein, 269 aa, synthetic construct
MAQRKLASVIGAALAASAVLVGLMTPATAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 84 - protein, 269 aa, synthetic construct
MSLSNKAKGIVAVIIVAWIVAMAGMVAYAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 85 - protein, 276 aa, synthetic construct
MTRALIALAVSGALLSSMTPAVAQPQNPDDAAIAQAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 86 - protein, 267 aa, synthetic construct
MRKFRNTAIALVSAAAITLGGVTAATAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 87 - protein, 267 aa, synthetic construct
MSLRRSTLTLVTASAVALSVFTPVAQAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 88 - protein, 271 aa, synthetic construct
MRKGISRVLSVAVASSIGFGTVLTGTGIAAAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 89 - protein, 280 aa, synthetic construct
MNFSLVHLRENVRRVSVTVAIGAGALLISGPFFTAHTAEAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 90 - protein, 266 aa, synthetic construct
MQGMKRKMVAAALATALLFGAAPAHAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 91 - protein, 276 aa, synthetic construct
MKVVEVKKRNLLVAPLTASLVFCNLAVAANAVEVEAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 92 - protein, 269 aa, synthetic construct
MSFKRRSLAITCAITSSIVLAGISGTAAAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 93 - protein, 288 aa, synthetic construct
MKIKKSASALSRSMRIGIATITSTAMLGGVLVAVPAHPLLPTTAVAQAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 94 - protein, 277 aa, synthetic construct
MQSIRTTFRSLSRSAVIVGTVLALGIAGCSTASDEATGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 95 - protein, 277 aa, synthetic construct
MSVFTRAGEASRKLVALVVALATAAALMVVGQGTAQAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 96 - protein, 261 aa, synthetic construct
MKQVFLLFLAFLVLFPSNAHAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 97 - protein, 278 aa, synthetic construct
MKKIHMIPAALLIGSLALVSCAPSQPTQNAQTVSSVMTGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 98 - protein, 271 aa, synthetic construct
MNISKLGLRIAVTATATTTALALGGTHMAVGGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 99 - protein, 264 aa, synthetic construct
MKTIRTGMMTLAALAVLGTNVVSAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 100 - protein, 263 aa, synthetic construct
MKKVVNSVLASALALTVAPMAFAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 101 - protein, 268 aa, synthetic construct
MSISVRFKSLIALLMTVVFLLVPSSAFAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 102 - protein, 270 aa, synthetic construct
MKKRRVVNSVLLLLLLASALALTVAPMAFAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 103 - protein, 273 aa, synthetic construct
MFAKRFKTSLLPLFAGFLLLFHLVLAGPAAASAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 104 - protein, 270 aa, synthetic construct
MRKKTKNRLISSVLSTVVISSLLFPGAAGAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 105 - protein, 275 aa, synthetic construct
MKISMQKADFWKKAAISLLVFTMFFTLMMSETVFAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 106 - protein, 263 aa, synthetic construct
MKRLLSTLLIGIMLLTFAPSAFAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 107 - protein, 267 aa, synthetic construct
MKNMSCKLVVSVTLFFSFLTIGPLAHAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 108 - protein, 268 aa, synthetic construct
MKKVLMAFIICLSLILSVLAAPPSGAKAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 109 - protein, 264 aa, synthetic construct
MRSYIKVLTMCFLGLILFVPTALAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 110 - protein, 271 aa, synthetic construct
MKKGIIRFLLVSFVLFFALSTGITGVQAAPAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 111 - protein, 271 aa, synthetic construct
MKRRKFSSVVAAVLIFALIFSLFSPGTKAAAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 112 - protein, 265 aa, synthetic construct
MKTKTLFIFSAILTLSIFAPNETFAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 113 - protein, 266 aa, synthetic construct
MKRITINIITMFIAAAVISLTGTAEAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 114 - protein, 261 aa, synthetic construct
MKWMCSICCAAVLLAGGAAQAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 115 - protein, 266 aa, synthetic construct
MLTKRLLTIYIMLLGLIAWFPGAAQAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 116 - protein, 265 aa, synthetic construct
MRFTKVVGFLSVLGLAAVFPLTAQAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 117 - protein, 263 aa, synthetic construct
MKRFILVLSFLSIIVAYPIQTNAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 118 - protein, 268 aa, synthetic construct
MTKKAWFLPLVCVLLISGWLAPAASASAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 119 - protein, 266 aa, synthetic construct
MKRIRIPMTLALGAALTIAPLSFASAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 120 - protein, 267 aa, synthetic construct
MKKIVSILFMFGLVMGFSQFQPSTVFAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 121 - protein, 281 aa, synthetic construct
MVKSFRMKALIAGAAVAAAVSAGAVSDVPAAKVLQPTAAYAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 122 - protein, 269 aa, synthetic construct
MKRSISIFITCLLITLLTMGGMIASPASAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 123 - protein, 259 aa, synthetic construct
MKKLVLCVSILAVILSGVAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 124 - protein, 272 aa, synthetic construct
MKKKKTWKRFLHFSSAALAAGLIFTSAAPAEAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 125 - protein, 267 aa, synthetic construct
MKKSQYFIVFICFFVLFSVHPIAAAAAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 126 - protein, 266 aa, synthetic construct
MKKKIVAGLAVSAVVGSSMAAAPAEAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 127 - protein, 266 aa, synthetic construct
MFNRLFRVCFLAALIMAFTLPNSVYAGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 128 - DNA, 57 bp, synthetic construct
  source 1..57: mol_type = other DNA
caatttcacacaggaaacagaattaagcttgcatgcctgcaggaaggagatatagat

SEQ ID NO: 129 - DNA, 36 bp, synthetic construct
  source 1..36: mol_type = other DNA
ggtagtcccgggctgccagggcccagaggggaacaa

SEQ ID NO: 130 - protein, 537 aa, Glaesserella parasuis
GPTGPQGPQGTPGTPGQKGDKGDPGPAGPKGDAGPKGDTGPRGEAGPAGPAGPRGPAGERGPAGETGPAGEPGKQGPKGEQGAPGPTGPRGERGEAGPAGAAGPAGPQGTPGPAGPRGEPGPKGEPGIQGPKGDKGDTGQKGEAGPAGPRGPEGPAGAKGEQGPRGEQGLPGVAGPKGDRGDVGPMGPVGPTGPQGAPGPAGPKGEAGAKGDRGDRGETGPEGPQGPRGEQGLRGEQGPAGPTGPRGEQGPEGKQGIQGPAGPAGPAGAQGIQGPKGNDGAPGARGEKGETGPAGPAGPRGPAGERGETGPAGPVGPAGPKGEIGPAGPTGAKGEPGPRGEQGIQGPAGPVGPAGPQGATGPTGPAGEPGKQGPRGEQGAPGPAGPKGEAGAKGDKGDTGEAGPMGPQGPAGAAGPAGPAGERGEQGPRGDKGETGPAGPAGAKGEPGPRGEQGIQGPAGPTGPQGPQGTAGIQGPKGDRGETGPAGAAGPVGPAGPRGEQGPKGEQGIQGPTGPTGPQGPQGTAGAQGPKGERGNV

SEQ ID NO: 131 - DNA, 1671 bp, synthetic construct
  source 1..1671: mol_type = other DNA
acctgcgcacgctggtagtcccgggctgccaggtccaaccggtccacagggtccacaaggcaccccaggcactccaggccagaagggcgataagggcgacccaggtccagcaggcccaaagggtgatgccggtccaaagggcgacaccggaccacgcggtgaagctggcccagcaggtcctgcaggtccacgtggcccagctggtgaacgcggtccagctggcgaaaccggtcctgccggtgaaccaggcaagcagggtcctaagggtgaacagggtgcacctggtccaactggccctcgcggtgaacgtggcgaggctggtcctgctggcgcagctggccctgccggaccacaaggtactccaggtccggctggacctcgtggcgaaccaggaccaaaaggcgagccaggtatccagggtccgaaaggcgacaaaggcgataccggtcagaaaggtgaggcaggccctgcgggtcctcgcggtcctgaaggaccagcaggcgctaaaggcgaacaaggcccacgcggagaacagggtctgccaggcgtggctggccctaagggcgatcgcggtgatgtgggtccaatgggtccagttggccctaccggtcctcaaggcgcacctggaccagcgggtccaaaaggcgaagcaggcgcaaagggcgatcgtggcgaccgtggtgaaaccggaccagaaggcccacagggacctcgcggagagcaaggcctgcgtggcgagcaaggaccggcaggtcctaccggaccgcgtggcgaacagggtccagaaggtaagcagggcatccagggtccagctggtccggctggccctgctggtgcacagggcattcagggaccaaagggcaacgatggcgcaccaggcgcacgtggcgaaaaaggtgaaactggtcccgcaggtccggctggcccacgcggaccggctggtgaacgtggtgaaactggccctgcaggccctgttggccctgcaggtcctaagggcgaaatcggtccagcaggtccgaccggtgccaagggcgaacctggtcctcgtggtgagcagggtattcaaggcccagcgggacccgtcggtcccgcaggaccacaaggtgcaaccggtcctaccggtccagcaggcgagccaggcaaacagggaccacgtggcgaacaaggcgcaccaggtcctgctggccctaaaggtgaagctggcgctaagggtgacaagggtgataccggtgaagcaggccctatgggtccacagggaccagcgggtgcagcaggcccagcgggtcctgccggtgagcgtggtgaacaaggccctcgtggcgataagggtgagactggaccggctggcccagcgggtgctaagggtgaaccaggtccacgcggtgaacaaggcatccaaggtcctgcaggccctactggtccacaaggcccacagggcaccgctggcattcaaggccctaagggtgaccgtggcgagactggcccagctggtgctgcgggtcctgttggtccagcgggtccacgcggagagcagggaccaaaaggtgaacaaggtattcaaggaccaaccggtccgactggtcctcagggaccacagggaactgcaggcgcacaaggaccgaagggcgaacgcggaaacgtgtaagaattcagcttggctgttttggcgga

SEQ ID NO: 132 - DNA, 7381 bp, synthetic construct
  source 1..7381: mol_type = other DNA
agaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattaagcttgcatgcctgcaggaaggagatatagatatgcagggtatgaaacggaagatggtcgcagcggcgctggcaacagcgcttctttttggggctgcacctgcgcacgctggtagtcccgggctgccagggcccagaggggaacaaggaccaacaggtccaaccggacctgctggtccacgaggtctacaaggtctacaaggtctacaaggtgaaagaggggaacaaggaccaacaggtcccgctggtccacgaggtctacaaggtgaaagaggggaacaaggaccaacaggtctcgctggtaaagccggtgaagctggagccaaaggcgaaaccggccccgctggtccacagggtccacgtggtgaacaaggcccgcaaggtcttccaggtaaagatggtgaagctggtgctcaaggcccagcaggtccaatgggtcctgctggtgagcgaggtgaaaaaggagaacctggtacccaaggcgctaaaggtgatcgcggtgaaaccggtccagtaggtccacgtggtgagcgaggcgaagccggtcccgctggaaaagatggtgaacgtggtccagtaggtccagctggtaaggacggccaaaacggccaagatggtcttccaggtaaagacggtaaggacggccaaaacggtaaagatggtcttccaggtaaagacggtaaggacggccaaaacggtaaagatggtcttccaggtaaagacggtaaggacggtcaagatggtaaagacggcctcccaggtaaagacggtaaagatggcctcccaggtaaggacggtaaggacggtcaaccaggtaaaccgggtaaatattaagaattcagcttggctgttttggcggatgag

SEQ ID NO: 133 - protein, 569 aa, synthetic construct
MQGMKRKMVAAALATALLFGAAPAHAGSPGLPGPTGPQGPQGTPGTPGQKGDKGDPGPAGPKGDAGPKGDTGPRGEAGPAGPAGPRGPAGERGPAGETGPAGEPGKQGPKGEQGAPGPTGPRGERGEAGPAGAAGPAGPQGTPGPAGPRGEPGPKGEPGIQGPKGDKGDTGQKGEAGPAGPRGPEGPAGAKGEQGPRGEQGLPGVAGPKGDRGDVGPMGPVGPTGPQGAPGPAGPKGEAGAKGDRGDRGETGPEGPQGPRGEQGLRGEQGPAGPTGPRGEQGPEGKQGIQGPAGPAGPAGAQGIQGPKGNDGAPGARGEKGETGPAGPAGPRGPAGERGETGPAGPVGPAGPKGEIGPAGPTGAKGEPGPRGEQGIQGPAGPVGPAGPQGATGPTGPAGEPGKQGPRGEQGAPGPAGPKGEAGAKGDKGDTGEAGPMGPQGPAGAAGPAGPAGERGEQGPRGDKGETGPAGPAGAKGEPGPRGEQGIQGPAGPTGPQGPQGTAGIQGPKGDRGETGPAGAAGPVGPAGPRGEQGPKGEQGIQGPTGPTGPQGPQGTAGAQGPKGERGNV

SEQ ID NO: 134 - DNA, 8290 bp, synthetic construct
  source 1..8290: mol_type = other DNA
agaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattaagcttgcatgcctgcaggaaggagatatagatatgcagggtatgaaacggaagatggtcgcagcggcgctggcaacagcgcttctttttggggctgcacctgcgcacgctggtagtcccgggctgccaggtccaaccggtccacagggtccacaaggcaccccaggcactccaggccagaagggcgataagggcgacccaggtccagcaggcccaaagggtgatgccggtccaaagggcgacaccggaccacgcggtgaagctggcccagcaggtcctgcaggtccacgtggcccagctggtgaacgcggtccagctggcgaaaccggtcctgccggtgaaccaggcaagcagggtcctaagggtgaacagggtgcacctggtccaactggccctcgcggtgaacgtggcgaggctggtcctgctggcgcagctggccctgccggaccacaaggtactccaggtccggctggacctcgtggcgaaccaggaccaaaaggcgagccaggtatccagggtccgaaaggcgacaaaggcgataccggtcagaaaggtgaggcaggccctgcgggtcctcgcggtcctgaaggaccagcaggcgctaaaggcgaacaaggcccacgcggagaacagggtctgccaggcgtggctggccctaagggcgatcgcggtgatgtgggtccaatgggtccagttggccctaccggtcctcaaggcgcacctggaccagcgggtccaaaaggcgaagcaggcgcaaagggcgatcgtggcgaccgtggtgaaaccggaccagaaggcccacagggacctcgcggagagcaaggcctgcgtggcgagcaaggaccggcaggtcctaccggaccgcgtggcgaacagggtccagaaggtaagcagggcatccagggtccagctggtccggctggccctgctggtgcacagggcattcagggaccaaagggcaacgatggcgcaccaggcgcacgtggcgaaaaaggtgaaactggtcccgcaggtccggctggcccacgcggaccggctggtgaacgtggtgaaactggccctgcaggccctgttggccctgcaggtcctaagggcgaaatcggtccagcaggtccgaccggtgccaagggcgaacctggtcctcgtggtgagcagggtattcaaggcccagcgggacccgtcggtcccgcaggaccacaaggtgcaaccggtcctaccggtccagcaggcgagccaggcaaacagggaccacgtggcgaacaaggcgcaccaggtcctgctggccctaaaggtgaagctggcgctaagggtgacaagggtgataccggtgaagcaggccctatgggtccacagggaccagcgggtgcagcaggcccagcgggtcctgccggtgagcgtggtgaacaaggccctcgtggcgataagggtgagactggaccggctggcccagcgggtgctaagggtgaaccaggtccacgcggtgaacaaggcatccaaggtcctgcaggccctactggtccacaaggcccacagggcaccgctggcattcaaggccctaagggtgaccgtggcgagactggcccagctggtgctgcgggtcctgttggtccagcgggtccacgcggagagcagggaccaaaaggtgaacaaggtattcaaggaccaaccggtccgactggtcctcagggaccacagggaactgcaggcgcacaaggaccgaagggcgaacgcggaaacgtgtaagaattcagcttggctgttttggcggatgag

SEQ ID NO: 135 - protein, 528 aa, Glaesserella parasuis
GRDGAPGPAGPRGERGEPGAPGPMGPQGPQGKEGPVGPAGATGQTGPAGPRGPKGDPGQKGDAGLQGPAGERGERGETGERGPVGPQGPRGDKGEQGDQGPRGPEGPQGPKGEPGIQGLRGEAGPPGPAGPIGPVGPAGDKGDKGDTGPVGPAGPIGPAGPAGPAGPVGPVGPQGAKGEQGPRGERGEQGLQGATGPAGPPGPAGARGEAGPRGERGEPGLAGPTGPQGAPGPVGPAGAAGPAGPRGPKGDPGQKGDAGLQGPAGERGERGPEGPQGPRGERGEQGLQGATGPAGPAGAPGVAGPKGDRGEAGPKGEAGPTGPRGERGPAGDKGDTGPMGPPGPAGARGEAGPKGEAGPAGAKGDQGPPGERGERGPEGPRGETGPAGAAGAKGEQGPKGDAGPAGPAGATGPMGPAGPRGPAGAPGPAGERGPAGEQGPAGPRGPKGDPGQKGDAGLQGPKGDAGLQGPKGDAGPPGPVGPAGPRGEKGEPGSRGEKGEPGPAGAKGDQGPPGERGERGPAGPAGPR

SEQ ID NO: 136 - DNA, 1644 bp, synthetic construct
  source 1..1644: mol_type = other DNA
acctgcgcacgctggtagtcccgggctgccaggtcgtgatggcgcaccaggtccagcaggcccacgcggtgaacgtggcgaaccaggtgctccaggtccaatgggtccacagggtcctcagggcaaagaaggcccagttggcccagcaggcgcaaccggtcaaaccggtccagctggtccacgtggcccaaagggcgatccaggccagaagggcgacgctggcttgcagggtcctgctggcgaacgcggagaacgcggtgagactggtgaacgcggtcctgttggcccacaaggcccacgtggcgataagggcgaacagggcgatcagggtccacgcggtccagaaggtcctcaaggcccaaaaggtgaaccaggcatccagggcctccgtggcgaagctggaccaccaggaccagccggtcctatcggtccagtcggtccggcaggcgacaagggtgacaagggcgataccggtcctgtgggtcctgccggtccaattggccctgctggtcccgcaggtccggctggaccagtgggtccagttggaccacagggtgcaaaaggcgaacaaggtcctcgcggagagcgtggcgagcagggtctgcaaggtgcaactggccctgcaggccctccaggacctgcaggcgctcgtggtgaagcaggtccgcgtggcgaacgtggtgaacctggcctggcaggccctaccggtccacaaggtgcccctggtcctgtcggacccgctggtgcggcaggcccagcgggacctcgtggtcctaaaggcgacccaggtcaaaagggtgatgcaggcttgcaaggcccagccggtgagcgtggtgaacgtggaccggaaggtccgcagggtccccgtggtgaacgcggagagcagggcctccaaggcgctactggtccggctggcccagctggcgctccaggtgttgcaggccctaagggtgatcgcggtgaagccggtccaaagggtgaagctggcccaaccggtcctcgcggtgaacgcggtccagcaggcgataagggcgacaccggtccaatgggacctccaggtcctgcgggtgcacgtggcgaggctggccctaaaggtgaggcaggtccagctggcgcaaagggtgaccagggtccaccaggcgaacgtggcgagcgtggcccagaaggaccacgcggagaaaccggtcctgcaggcgcagcaggcgctaaaggcgagcaaggaccaaaaggcgacgcaggcccagcaggtcccgcaggcgcaactggtcctatgggtccagctggccctcgtggtccggctggtgctcctggtcctgcaggcgaacgtggacctgcaggcgaacagggtccagcaggtcctcgcggtcctaagggtgaccctggccagaaaggtgatgctggactgcaaggaccgaagggtgatgccggtctgcagggtccaaaaggtgacgctggtcctccaggtccagtgggacccgcaggtcctcgtggtgagaagggcgaaccaggctctcgcggtgaaaagggcgagcctggtcctgccggtgcaaaaggtgatcaaggcccaccaggtgaacgcggtgagcgtggtccggcaggtccagctggtcctcgttaagaattcagcttggctgttttggcgga

SEQ ID NO: 137 - protein, 560 aa, synthetic construct
MQGMKRKMVAAALATALLFGAAPAHAGSPGLPGRDGAPGPAGPRGERGEPGAPGPMGPQGPQGKEGPVGPAGATGQTGPAGPRGPKGDPGQKGDAGLQGPAGERGERGETGERGPVGPQGPRGDKGEQGDQGPRGPEGPQGPKGEPGIQGLRGEAGPPGPAGPIGPVGPAGDKGDKGDTGPVGPAGPIGPAGPAGPAGPVGPVGPQGAKGEQGPRGERGEQGLQGATGPAGPPGPAGARGEAGPRGERGEPGLAGPTGPQGAPGPVGPAGAAGPAGPRGPKGDPGQKGDAGLQGPAGERGERGPEGPQGPRGERGEQGLQGATGPAGPAGAPGVAGPKGDRGEAGPKGEAGPTGPRGERGPAGDKGDTGPMGPPGPAGARGEAGPKGEAGPAGAKGDQGPPGERGERGPEGPRGETGPAGAAGAKGEQGPKGDAGPAGPAGATGPMGPAGPRGPAGAPGPAGERGPAGEQGPAGPRGPKGDPGQKGDAGLQGPKGDAGLQGPKGDAGPPGPVGPAGPRGEKGEPGSRGEKGEPGPAGAKGDQGPPGERGERGPAGPAGPR

SEQ ID NO: 138 - DNA, 8263 bp, synthetic construct
  source 1..8263: mol_type = other DNA
agaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattaagcttgcatgcctgcaggaaggagatatagatatgcagggtatgaaacggaagatggtcgcagcggcgctggcaacagcgcttctttttggggctgcacctgcgcacgctggtagtcccgggctgccaggtcgtgatggcgcaccaggtccagcaggcccacgcggtgaacgtggcgaaccaggtgctccaggtccaatgggtccacagggtcctcagggcaaagaaggcccagttggcccagcaggcgcaaccggtcaaaccggtccagctggtccacgtggcccaaagggcgatccaggccagaagggcgacgctggcttgcagggtcctgctggcgaacgcggagaacgcggtgagactggtgaacgcggtcctgttggcccacaaggcccacgtggcgataagggcgaacagggcgatcagggtccacgcggtccagaaggtcctcaaggcccaaaaggtgaaccaggcatccagggcctccgtggcgaagctggaccaccaggaccagccggtcctatcggtccagtcggtccggcaggcgacaagggtgacaagggcgataccggtcctgtgggtcctgccggtccaattggccctgctggtcccgcaggtccggctggaccagtgggtccagttggaccacagggtgcaaaaggcgaacaaggtcctcgcggagagcgtggcgagcagggtctgcaaggtgcaactggccctgcaggccctccaggacctgcaggcgctcgtggtgaagcaggtccgcgtggcgaacgtggtgaacctggcctggcaggccctaccggtccacaaggtgcccctggtcctgtcggacccgctggtgcggcaggcccagcgggacctcgtggtcctaaaggcgacccaggtcaaaagggtgatgcaggcttgcaaggcccagccggtgagcgtggtgaacgtggaccggaaggtccgcagggtccccgtggtgaacgcggagagcagggcctccaaggcgctactggtccggctggcccagctggcgctccaggtgttgcaggccctaagggtgatcgcggtgaagccggtccaaagggtgaagctggcccaaccggtcctcgcggtgaacgcggtccagcaggcgataagggcgacaccggtccaatgggacctccaggtcctgcgggtgcacgtggcgaggctggccctaaaggtgaggcaggtccagctggcgcaaagggtgaccagggtccaccaggcgaacgtggcgagcgtggcccagaaggaccacgcggagaaaccggtcctgcaggcgcagcaggcgctaaaggcgagcaaggaccaaaaggcgacgcaggcccagcaggtcccgcaggcgcaactggtcctatgggtccagctggccctcgtggtccggctggtgctcctggtcctgcaggcgaacgtggacctgcaggcgaacagggtccagcaggtcctcgcggtcctaagggtgaccctggccagaaaggtgatgctggactgcaaggaccgaagggtgatgccggtctgcagggtccaaaaggtgacgctggtcctccaggtccagtgggacccgcaggtcctcgtggtgagaagggcgaaccaggctctcgcggtgaaaagggcgagcctggtcctgccggtgcaaaaggtgatcaaggcccaccaggtgaacgcggtgagcgtggtccggcaggtccagctggtcctcgttaagaattcagcttggctgttttggcggatgag

SEQ ID NO: 139 - protein, 771 aa, Streptosporangium roseum
GPEGAAGPRGLTGPVGPRGPKGDTGAQGPKGDTGPQGPQGRQGPQGAEGSRGPKGDTGEQGRPGVAGPAGPQGEPGKRGDTGAQGPKGDTGPQGPQGRQGPQGADGPRGDTGPQGPKGDTGAQGSQGSQGPQGSQGPRGPKGDTGGTGPQGPQGPTGETGATGETGPQGLPGDTGPQGPQGDTGPQGPKGDTGDQGPQGPQGDTGDQGPKGDTGDTGPQGEQGPQGLPGDTGPQGPQGDTGPQGPKGDTGDQGPQGPQGDTGDQGPRGDTGPQGPKGDTGDQGPQGPQGDTGAQGPKGDTGDTGPQGETGPQGDTGPQGEQGEQGDTGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGDTGPQGEAGPQGDTGPQGEAGPQGEAGPQGEAGPQGDTGPQGEAGPQGDAGPQGEAGPQGDTGPQGEAGPQGDTGPQGEAGPQGDTGPQGEAGPQGDAGPQGDTGPQGEAGPQGEAGPQGEAGPQGEQGPQGDTGPQGPQGDAGIV

SEQ ID NO: 140 - DNA, 2373 bp, synthetic construct
  source 1..2373: mol_type = other DNA
acctgcgcacgctggtagtcccgggctgccaggtccagaaggcgcagcaggtccacgcggtctgaccggtccagttggccctcgcggtccaaagggcgataccggtgcacaaggcccaaaaggtgacaccggtccacagggtccacaaggtcgccagggtcctcagggtgccgaaggctcccgtggtcctaagggtgatactggcgaacagggtcgcccaggcgtggcaggtccagcaggaccacagggcgaaccaggcaagcgtggcgacactggcgctcagggtcctaaaggcgacaccggacctcaaggccctcagggtcgtcaaggtcctcaaggcgcagatggcccacgtggtgataccggaccgcagggaccgaaaggcgatactggtgcccagggctcccaaggctcccagggaccacaaggctctcaaggcccacgcggaccgaagggtgacactggtggcactggccctcaaggaccccaaggtccaaccggtgaaaccggtgctaccggtgagactggcccacagggcctgccaggcgataccggtcctcagggaccacagggtgataccggtccgcaaggccctaagggcgacactggtgaccagggtccgcagggacctcaaggtgacactggcgatcaaggaccaaaaggtgatactggcgataccggaccacagggcgagcagggtccacagggccttccgggtgatactggaccacaaggtccgcagggcgatactggaccccagggtccaaagggtgacaccggtgatcagggacctcagggtccccaaggcgataccggtgatcaaggtccacgtggcgatactggcccacaaggaccaaaaggcgacactggtgatcaaggccctcaaggtccacagggtgacactggcgcacagggacccaagggcgatactggcgacaccggtccgcagggtgaaaccggtcctcagggcgacaccggtccacaaggcgaacagggcgaacaaggcgacactggcccacagggtgaagcaggccctcagggtgaagctggtccgcaaggcgaagcaggcccacaaggcgatactggtcctcaaggcgaggctggtccccagggcgaagcgggtcctcaaggtgaagccggaccgcaaggtgaggctggaccacaaggcgaggcaggaccgcagggtgacaccggaccacaaggtgaagcgggaccccagggcgaagccggaccacaaggcgaagctggtcctcagggtgaagctggcccacaaggtgaggcaggtccccaaggtgatactggtccgcagggtgaagcgggaccacagggtgaagcaggtcctcaaggcgaggcaggacctcaaggtgaagcaggtccacaaggtgaagccggtccacagggcgataccggacctcaaggtgaggcaggcccacagggcgaagctggcccacagggcgaagcaggaccgcaaggtgaggcaggtcctcagggtgaagcaggacctcagggcgataccggtccgcagggcgaagctggacctcaaggcgaggctggcccacaaggtgaggctggccctcaaggcgaagccggtccacaaggcgaggcaggtccgcaaggcgacactggaccgcaaggcgaagcgggtccgcagggcgaagcaggtccccaaggtgaagcaggccctcaaggcgaggctggtccacagggtgaagccggtccgcaaggtgatactggcccacagggcgaagccggtccgcagggtgaagcgggtccacaaggcgaagctggccctcaaggtgaggcaggaccgcaaggcgaagcgggacctcaaggtgataccggtcctcaaggcgaagcaggaccgcagggcgaagctggcccacaaggtgaagcgggtcctcagggtgacactggtccacagggcgaagctggtccacaaggcgataccggtccacaaggtgaagctggcccacagggtgaagccggaccacagggcgaagcgggtccccaaggtgacactggcccacaaggcgaggcaggcccacagggtgatgcgggaccccaaggcgaggcaggtccacagggcgacaccggtcctcagggcgaagcaggtccacagggtgatactggccctcaaggcgaagctggccctcagggcgatactggccctcagggtgaagccggacctcaaggtgatgcaggccctcaaggtgataccggaccacagggtgaagctggaccgcaaggtgaagccggtccgcaaggcgaagccggaccacagggtgagcaaggaccgcagggcgacactggtcctcaaggtccacaaggcgacgcaggcatcgtgtaagaattcagcttggctgttttggcgga

SEQ ID NO: 141 - protein, 803 aa, synthetic construct
MQGMKRKMVAAALATALLFGAAPAHAGSPGLPGPEGAAGPRGLTGPVGPRGPKGDTGAQGPKGDTGPQGPQGRQGPQGAEGSRGPKGDTGEQGRPGVAGPAGPQGEPGKRGDTGAQGPKGDTGPQGPQGRQGPQGADGPRGDTGPQGPKGDTGAQGSQGSQGPQGSQGPRGPKGDTGGTGPQGPQGPTGETGATGETGPQGLPGDTGPQGPQGDTGPQGPKGDTGDQGPQGPQGDTGDQGPKGDTGDTGPQGEQGPQGLPGDTGPQGPQGDTGPQGPKGDTGDQGPQGPQGDTGDQGPRGDTGPQGPKGDTGDQGPQGPQGDTGAQGPKGDTGDTGPQGETGPQGDTGPQGEQGEQGDTGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGEAGPQGEAGPQGDTGPQGEAGPQGEAGPQGEAGPQGDTGPQGEAGPQGDTGPQGEAGPQGEAGPQGEAGPQGDTGPQGEAGPQGDAGPQGEAGPQGDTGPQGEAGPQGDTGPQGEAGPQGDTGPQGEAGPQGDAGPQGDTGPQGEAGPQGEAGPQGEAGPQGEQGPQGDTGPQGPQGDAGIV

SEQ ID NO: 142 - DNA, 8992 bp, synthetic construct
  source 1..8992: mol_type = other DNA
agaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattaagcttgcatgcctgcaggaaggagatatagatatgcagggtatgaaacggaagatggtcgcagcggcgctggcaacagcgcttctttttggggctgcacctgcgcacgctggtagtcccgggctgccaggtccagaaggcgcagcaggtccacgcggtctgaccggtccagttggccctcgcggtccaaagggcgataccggtgcacaaggcccaaaaggtgacaccggtccacagggtccacaaggtcgccagggtcctcagggtgccgaaggctcccgtggtcctaagggtgatactggcgaacagggtcgcccaggcgtggcaggtccagcaggaccacagggcgaaccaggcaagcgtggcgacactggcgctcagggtcctaaaggcgacaccggacctcaaggccctcagggtcgtcaaggtcctcaaggcgcagatggcccacgtggtgataccggaccgcagggaccgaaaggcgatactggtgcccagggctcccaaggctcccagggaccacaaggctctcaaggcccacgcggaccgaagggtgacactggtggcactggccctcaaggaccccaaggtccaaccggtgaaaccggtgctaccggtgagactggcccacagggcctgccaggcgataccggtcctcagggaccacagggtgataccggtccgcaaggccctaagggcgacactggtgaccagggtccgcagggacctcaaggtgacactggcgatcaaggaccaaaaggtgatactggcgataccggaccacagggcgagcagggtccacagggccttccgggtgatactggaccacaaggtccgcagggcgatactggaccccagggtccaaagggtgacaccggtgatcagggacctcagggtccccaaggcgataccggtgatcaaggtccacgtggcgatactggcccacaaggaccaaaaggcgacactggtgatcaaggccctcaaggtccacagggtgacactggcgcacagggacccaagggcgatactggcgacaccggtccgcagggtgaaaccggtcctcagggcgacaccggtccacaaggcgaacagggcgaacaaggcgacactggcccacagggtgaagcaggccctcagggtgaagctggtccgcaaggcgaagcaggcccacaaggcgatactggtcctcaaggcgaggctggtccccagggcgaagcgggtcctcaaggtgaagccggaccgcaaggtgaggctggaccacaaggcgaggcaggaccgcagggtgacaccggaccacaaggtgaagcgggaccccagggcgaagccggaccacaaggcgaagctggtcctcagggtgaagctggcccacaaggtgaggcaggtccccaaggtgatactggtccgcagggtgaagcgggaccacagggtgaagcaggtcctcaaggcgaggcaggacctcaaggtgaagcaggtccacaaggtgaagccggtccacagggcgataccggacctcaaggtgaggcaggcccacagggcgaagctggcccacagggcgaagcaggaccgcaaggtgaggcaggtcctcagggtgaagcaggacctcagggcgataccggtccgcagggcgaagctggacctcaaggcgaggctggcccacaaggtgaggctggccctcaaggcgaagccggtccacaaggcgaggcaggtccgcaaggcgacactggaccgcaaggcgaagcgggtccgcagggcgaagcaggtccccaaggtgaagcaggccctcaaggcgaggctggtccacagggtgaagccggtccgcaaggtgatactggcccacagggcgaagccggtccgcagggtgaagcgggtccacaaggcgaagctggccctcaaggtgaggcaggaccgcaaggcgaagcgggacctcaaggtgataccggtcctcaaggcgaagcaggaccgcagggcgaagctggcccacaaggtgaagcgggtcctcagggtgacactggtccacagggcgaagctggtccacaaggcgataccggtccacaaggtgaagctggcccacagggtgaagccggaccacagggcgaagcgggtccccaaggtgacactggcccacaaggcgaggcaggcccacagggtgatgcgggaccccaaggcgaggcaggtccacagggcgacaccggtcctcagggcgaagcaggtccacagggtgatactggccctcaaggcgaagctggccctcagggcgatactggccctcagggtgaagccggacctcaaggtgatgcaggccctcaaggtgataccggaccacagggtgaagctggaccgcaaggtgaagccggtccgcaaggcgaagccggaccacagggtgagcaaggaccgcagggcgacactggtcctcaaggtccacaaggcgacgcaggcatcgtgtaagaattcagcttggctgttttggcggatgag